SEQUENCE LISTING (86 sequences)

SEQ ID NO: 1 - protein, 161 aa, Homo sapiens
MDDIYKAAVEQLTEEQKNEFKAAFDIFVLGAEDGCISTKELGKVMRMLGQNPTPEELQEMIDEVDEDGSGTVDFDEFLVMMVRCMKDDSKGKSEEELSDLFRMFDKNADGYIDLDELKIMLQATGETITEDDIEELMKDGDKNNDGRIDYDEFLEFMKGVE

SEQ ID NO: 2 - DNA, 483 bp, Homo sapiens
  source 1..483: mol_type = genomic DNA
atggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggag

SEQ ID NO: 3 - DNA, 10 bp, unidentified
  misc_feature 1..10: note = Kozak sequence
  source 1..10: mol_type = unassigned DNA
gccaccatgg

SEQ ID NO: 4 - DNA, 489 bp, synthetic construct
  misc_feature 1..489: note = polynucleotide encoding TNNC1 and Kozak sequence
  source 1..489: mol_type = other DNA
gccaccatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggag

SEQ ID NO: 5 - DNA, 13 bp, unidentified
  misc_feature 1..13: note = Alternative kozak sequence
  source 1..13: mol_type = genomic DNA
gccgccrccatgg

SEQ ID NO: 6-9 - skipped (4 entries)

SEQ ID NO: 10 - DNA, 10 bp, unidentified
  misc_feature 1..10: note = Alternative kozak sequence
  source 1..10: mol_type = unassigned DNA
gacaccatgg

SEQ ID NO: 11 - DNA, 141 bp, unidentified
  source 1..141: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 12 - DNA, 168 bp, unidentified
  source 1..168: mol_type = genomic DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 13 - DNA, 170 bp, unidentified
  source 1..170: mol_type = genomic DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 14 - DNA, 145 bp, unidentified
  source 1..145: mol_type = genomic DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 15 - DNA, 141 bp, unidentified
  source 1..141: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 16 - DNA, 168 bp, unidentified
  source 1..168: mol_type = genomic DNA
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 17 - DNA, 133 bp, unidentified
  source 1..133: mol_type = genomic DNA
aggaacccctagtgatggagactccctctctgcgcgctcgctcgctcactgaggccgggcgaccaaaggtcgcccgacgcccgggctttgcccgggcggcctcagtgagcgagcgagcgcgcagagagggagt

SEQ ID NO: 18 - DNA, 124 bp, synthetic construct
  misc_feature 1..124: note = Made in Lab - vector filler sequence
  source 1..124: mol_type = other DNA
gcggcaattcagtcgataactataacggtcctaaggtagcgatttaaatacgcgctctcttaaggtagccccgggacgcgtcaattgactacaaaccgagtatctgcagagggccctgcgtatg

SEQ ID NO: 19 - DNA, 84 bp, synthetic construct
  misc_feature 1..84: note = Made in Lab - vector filler sequence
  source 1..84: mol_type = other DNA
cttctgaggcggaaagaaccagatcctctcttaaggtagcatcgagatttaaattagggataacagggtaatggcgcgggccgc

SEQ ID NO: 20 - DNA, 63 bp, synthetic construct
  misc_feature 1..63: note = Made in Lab - vector filler sequence
  source 1..63: mol_type = other DNA
gttacccaggctggagtgcagtggcacatttctgctcactgcaacctcctcctccctgggttc

SEQ ID NO: 21 - DNA, 771 bp, synthetic construct
  misc_feature 1..771: note = Made in Lab - MHCK7 promoter
  source 1..771: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccag

SEQ ID NO: 22 - DNA, 502 bp, Homo sapiens
  source 1..502: mol_type = genomic DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagt

SEQ ID NO: 23 - DNA, 544 bp, Homo sapiens
  source 1..544: mol_type = genomic DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcag

SEQ ID NO: 24 - DNA, 5464 bp, Mus musculus
  source 1..5464: mol_type = genomic DNA
ggtaccggatcctgcaaggtcacacaagggtctccacccaccaggtgccctagtctcaatttcagtttccatgccttgttctcacaatgctggcctccccagagctaatttggactttgtttttatttcaaaagggcctgaatgaggagtagatcttgtgctacccagctctaagggtgcccgtgaagccctcagacctggagcctttgcaacagccctttaggtggaagcagaataaagcaattttccttaaagccaaaatcctgcctctagactcttcttctctgacctcggtccctgggctctagggtggggaggtggggcttggaagaagaaggtggggaagtggcaaaagccgatccctagggccctgtgaagttcggagccttccctgtacagcactggctcatagatcctcctccagccaaacatagcaagaagtgatacctcctttgtgacttccccaggcccagtacctgtcaggttgaaacaggatttagagaagcctctgaactcacctgaactctgaagctcatccaccaagcaagcacctaggtgccactgctagttagtatcctacgctgataatatgcagagctgggccacagaagtcctggggtgtaggaactgaccagtgacttttcagtcggcaaaggtatgaccccctcagcagatgtagtaatgtccccttagatcccatcccaggcaggtctctaagaggacatgggatgagagatgtagtcatgtggcattccaaacacagctatccacagtgtcccttgccccttccacttagccaggaggacagtaaccttagcctatctttcttcctccccatcctcccaggacacaccccctggtctgcagtattcatttcttccttcacgtcccctctgtgacttccatttgcaaggcttttgacctctgcagctgctggaagatagagtttggccctaggtgtggcaagccatctcaagagaaagcagacaacagggggaccagattttggaaggatcaggaactaaatcactggcgggcctgggggtagaaaaaagagtgagtgagtccgctccagctaagccaagctagtccccgagatactctgccacagctgggctgctcggggtagctttaggaatgtgggtctgaaagacaatgggattggaagacatctctttgagtctcccctcaaccccacctacagacacactcgtgtgtggccagactcctgttcaacagccctctgtgttctgaccactgagctaggcaaccagagcatgggccctgtgctgaggatgaagagttggttaccaatagcaaaaacagcaggggagggagaacagagaacgaaataaggaaggaagaaggaaaggccagtcaatcagatgcagtcagaagagatgggaagccaacacacagcttgagcagaggaaacagaaaagggagagattctgggcataaggaggccacagaaagaagagcccaggccccccaagtctcctctttataccctcatcccgtctcccaattaagcccactcttcttcctagatcagacctgagctgcagcgaagagacccgtagggaggatcacactggatgaaggagatgtgtggagaagtccagggaacctaagagccagagcctaaaagagcaagagataaaggtgcttcaaaggtggccaggctgtgcacacagagggtcgaggactggtggtagagcctcaagataaggatgatgctcagaatgggcggggggggggattctgggggggggagagagaaggtgagaaggagcctggaacagagaatctggaagcgctggaaacgataccataaagggaagaacccaggctacctttagatgtaaatcatgaaagacagggagaagggaagctggagagagtagaaggaccccggggcaagacattgaagcaaggacaagccaggttgagcgctccgtgaaatcagcctgctgaaggcagagccctggtatgagcaccagaacagcagaggctagggttaatgtcgagacagggaacagaaggtagacacaggaacagacagagacgggggagccaggtaacaaaggaatggtccttctcacctgtggccagagcgtccatctgtgtccacatactctagaatgttcatcagactgcagggctggcttgggaggcagctggaaagagtatgtgagagccaggggagacaagggggcctaggaaaggaagaagagggcaaaccaggccacacaagagggcagagcccagaactgagttaactccttccttgttgcatcttccataggaggcagtgggaactctgtgaccaccatcccccatgagcccccactacccataccaagtttggcctgagtggcattctaggttccctgaggacagagcctggcctttgtctcttggacctgacccaagctgacccaatgttctcagtaccttatcatgccctcaagagcttgagaaccaggcagtgacatattaggccatgggctaaccctggagcttgcacacaggagcctcaagtgacctccagggacacagctgcagacaggtggcctttatccccaaagagcaaccatttggcataggtggctgcaaatgggaatgcaaggttgaatcaggtcccttcaagaatactgcatgcaagacctaagacccctggagagaggggtatgctcctgcccccacccaccataaggggagtgaactatcctagggggctggcgaccttggggagacaccacattactgagagtgctgagcccagaaaaactgaccgccctgtgtcctgcccacctccacactctagagctatattgagaggtgacagtagatagggtgggagctggtagcagggagagtgttcctgggtgtgagggtgtaggggaaagccagagcaggggagtctggctttgtctcctgaacacaatgtctacttagttataacaggcatgacctgctaaagacccaacatctacgacctctgaaaagacagcagccctggaggacaggggttgtctctgagccttgggtgcttgatggtgccacaaaggagggcatgagtgtgagtataaggccccaggagcgttagagaagggcacttgggaaggggtcagtctgcagagcccctatccatggaatctggagcctggggccaactggtgtaaatctctgggcctgccaggcattcaaagcagcacctgcatcctctggcagcctggggaggcggaagggagcaaccccccacttataccctttctccctcagccccaggattaacacctctggccttcccccttcccacctcccatcaggagtggagggttgcagagggagggtaaaaacctacatgtccaaacatcatggtgcacgatatatggatcagtatgtgtagaggcaagaaaggaaatctgcaggcttaactgggttaatgtgtaaagtctgtgtgcatgtgtgtgtgtctgactgaaaacgggcatggctgtgcagctgttcagttctgtgcgtgaggttaccagactgcaggtttgtgtgtaaattgcccaaggcaaagtgggtgaatcccttccatggtttaaagagattggatgatggcctgcatctcaaggaccatggaaaatagaatggacactctatatgtgtctctaagctaaggtagcaaggtctttggaggacacctgtctagagatgtgggcaacagagactacagacagtatctgtacagagtaaggagagagaggagggggtgtagaattctcttactatcaaagggaaactgagtcgtgcacctgcaaagtggatgctctccctagacatcatgactttgtctctggggagccagcactgtggaacttcaggtctgagagagtaggaggctcccctcagcctgaagctatgcagatagccagggttgaaagggggaagggagagcctgggatgggagcttgtgtgttggaggcaggggacagatattaagcctggaagagaaggtgacccttacccagttgttcaactcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcctcctgtcacctccagagccaagggatcaaaggaggaggagccaggacaggagggaagtgggagggagggtcccagcagaggactccaaatttaggcagcaggcatatgggatgggatataaaggggctggagcactgagagctgtcagagatttctccaacccaggtaagagggagtttcgggtgggggctcttcacccacaccagacctctccccacctagaaggaaactgcctttcctggaagtggggttcaggccggtcagagatctgacagggtggccttccaccagcctgggaagttctcagtggcaggaggtttccacaagaaacactggatgccccttcccttacgctgtcttctccatcttcctcctggggatgctcctccccgtcttggtttatcttggctcttcgtcttcagcaagatttgccctgtgctgtccactccatctttctctactgtctccgtgccttgccttgccttcttgcgtgtccttcctttccacccatttctcacttcaccttttctccccttctcatttgtattcatccttccttccttccttccttccttccttccttccttccttccttcctttctcccttccttccttccttccttccttccttccttccttccttcctgtgtcagagtgctgagaatcacacctggggttcccacccttatgtaaacaatcttccagtgagccacagcttcagtgctgctgggtgctctcttaccttcctcaccccctggcttgtcctgttccatcctggtcaggatctctagattggtctcccagcctctgctactcctcttcctgcctgttcctctctctgtccagctgcgccactgtggtgcctcgttccagctgtggtccacattcttcaggattctctgaaaagttaaccaggtgagaatgtttcccctgtagacagcagatcacgattctcccggaagtcaggcttccagccctctctttctctgcccagctgcccggcactcttagcaaacctcaggcacccttaccccacatagacctctgacagagaagcaggcactttacatggagtcctggtgggagagccataggctacggtgtaaaagaggcagggaagtggtggtgtaggaaagtcaggacttcacatagaagcctagcccacaccagaaatgacagacagatccctcctatctcccccataagagtttgagtcgacccgcggccccgaattg

SEQ ID NO: 25 - DNA, 413 bp, Gallus gallus
  source 1..413: mol_type = genomic DNA
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtggga

SEQ ID NO: 26 - DNA, 1090 bp, Homo sapiens
  source 1..1090: mol_type = genomic DNA
ctctcagccctggaagtccttgctcacagccgaggcgccgagagcgcttgctctgcccagatctgcgcgagtctggcgcccgcgctctgaacggcgtcgctgcccagcccccttccccgggaggtgggagcggccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtcccccgcacagcggaaatctcccagtggcaccgagggggcgagggttaagtgggggggagggtgaccaccgcctcccacccttgccctgagtttgaatctctccaactcagccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagaca

SEQ ID NO: 27 - DNA, 253 bp, Homo sapiens
  source 1..253: mol_type = genomic DNA
gcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagtc

SEQ ID NO: 28 - DNA, 281 bp, Gallus gallus
  source 1..281: mol_type = genomic DNA
ggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggga

SEQ ID NO: 29 - DNA, 220 bp, Human betaherpesvirus 5
  source 1..220: mol_type = genomic DNA
tggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 30 - DNA, 583 bp, Human betaherpesvirus 5
  source 1..583: mol_type = genomic DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgt

SEQ ID NO: 31 - DNA, 508 bp, Human betaherpesvirus 5
  source 1..508: mol_type = genomic DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 32 - DNA, 573 bp, synthetic construct
  misc_feature 1..573: note = Made in lab - CAG promoter in part Human betaherpesvirus 5
  source 1..573: mol_type = other DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 33 - DNA, 580 bp, synthetic construct
  misc_feature 1..580: note = Made in lab - CAG promoter in part Human betaherpesvirus 5
  source 1..580: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 34 - DNA, 455 bp, Homo sapiens
  source 1..455: mol_type = genomic DNA
caacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaatagcatgcgtgaggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 35 - DNA, 281 bp, Homo sapiens
  source 1..281: mol_type = genomic DNA
acttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 36 - DNA, 293 bp, Human betaherpesvirus 5
  source 1..293: mol_type = genomic DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 37 - skipped

SEQ ID NO: 38 - DNA, 953 bp, Homo sapiens
  source 1..953: mol_type = genomic DNA
cgcgtccgcccgcgagcacagagcctcgcctttgccgatccgccgcccgtccacacccgccgccaggtaagcccggccagccgaccggggcatgcggccgcggcccttcgcccgtgcagagccgccgtctgggccgcagcggggggcgcatggggcggaaccggaccgccgtggggggcgcgggagaagcccctgggcctccggagatgggggacaccccacgccagttcgcaggcgcgaggccgcgctcgggcgggcgcgctccgggggtgccgctctcggggcgggggcaaccggcggggtctttgtctgagccgggctcttgccaatggggatcgcacggtgggcgcggcgtagcccccgtcaggcccggtgggggctggggcgccatgcgcgtgcgcgctggtcctttgggcgctaactgcgtgcgcgctgggaattggcgctaattgcgcgtgcgcgctgggactcaatggcgctaatcgcgcgtgcgttctggggcccgggcgcttgcgccacttcctgcccgagccgctggcgcccgagggtgtggccgctgcgtgcgcgcgcgcgacccggtcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcggcagcggctgctcttggcggccccgaggtgactatagccttcttttgtgtcttgatagttcgccagcctctgctaaccatgttcatgccttcttctttttcctacagctcctgggcaacgtgctggttattgtgctgtctcatcattttggcaaagaattc

SEQ ID NO: 39 - DNA, 1068 bp, synthetic construct
  misc_feature 1..1068: note = Made in Lab - Chicken beta-actin exon/intron plus rabbit globin intron
  source 1..1068: mol_type = other DNA
gtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 40 - DNA, 149 bp, synthetic construct
  misc_feature 1..149: note = Made in Lab - chimeric intron sequence
  source 1..149: mol_type = other DNA
ggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 41 - DNA, 126 bp, Homo sapiens
  source 1..126: mol_type = genomic DNA
agtctgcggtgggcagcggaggagtcgtgtcgtgcctgagagcgcagctgtgctcctgggcaccgcgcagtccgcccccgcggctcctggccagaccacccctaggaccccctgccccaagtcgca

SEQ ID NO: 42 - DNA, 121 bp, Human betaherpesvirus 5
  source 1..121: mol_type = genomic DNA
tcagatcgcctggagaggccatccacgctgttttgacctccatagtggacaccgggaccgatccagcctccgcggccgggaacggtgcattggaacgcggattccccgtgccaagagtgac

SEQ ID NO: 43 - DNA, 512 bp, synthetic construct
  misc_feature 1..512: note = Made in Lab - adenovirus derived enhancer element
  source 1..512: mol_type = other DNA
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccag

SEQ ID NO: 44 - DNA, 956 bp, Homo sapiens
  source 1..956: mol_type = genomic DNA
ctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacgtgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcag

SEQ ID NO: 45 - DNA, 939 bp, Homo sapiens
  source 1..939: mol_type = genomic DNA
gtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 46 - DNA, 83 bp, Homo sapiens
  source 1..83: mol_type = genomic DNA
tcagaagccccgggctcgtcagtcaaaccggttctctgtttgcactcggcagcacgggcaggcaagtggtccctaggttcggg

SEQ ID NO: 47 - DNA, 476 bp, Homo sapiens
  source 1..476: mol_type = genomic DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 48 - DNA, 196 bp, unidentified
  source 1..196: mol_type = genomic DNA
tctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 49 - DNA, 589 bp, synthetic construct
  misc_feature 1..589: note = Made in Lab - mutated woodchuck hepatitis regulatory element
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 50 - DNA, 588 bp, synthetic construct
  misc_feature 1..588: note = Made in Lab - mutated woodchuck hepatitis regulatory element
  source 1..588: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 51 - DNA, 755 bp, synthetic construct
  misc_feature 1..755: note = Made in Lab - mutated woodchuck hepatitis regulatory element
  source 1..755: mol_type = other DNA
ttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 52 - DNA, 12 bp, synthetic construct
  misc_feature 1..12: note = CAAX motif
  source 1..12: mol_type = other DNA
tgtgtgataatg

SEQ ID NO: 53 - DNA, 810 bp, Homo sapiens
  source 1..810: mol_type = genomic DNA
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 54 - DNA, 726 bp, Hepatitis B virus
  source 1..726: mol_type = genomic DNA
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 55 - DNA, 755 bp, synthetic construct
  misc_feature 1..755: note = Made in Lab - HepB derived enhancer element
  source 1..755: mol_type = other DNA
ttcctgtaaacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagctggactggagcaaacctcatcgggaccgacaattctgtcgtactctcccgcaagcactcaccgtttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 56 - DNA, 94 bp, Homo sapiens
  source 1..94: mol_type = genomic DNA
gctggagcctcggtagccgttcctcctgcccgctgggcctcccaacgggccctcctcccctccttgcaccggcccttcctggtctttgaataaa

SEQ ID NO: 57 - DNA, 2512 bp, synthetic construct
  misc_feature 1..2512: note = Made in Lab - AAV-MHCK7-TNNC1 vector construct
  source 1..2512: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagtagtcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 58 - DNA, 2136 bp, synthetic construct
  misc_feature 1..2136: note = Made in lab - AAV-hTnT-TNNC1 vector construct
  source 1..2136: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagtagtcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 59 - DNA, 2124 bp, synthetic construct
  misc_feature 1..2124: note = Made in lab - scAAV-hTNT-TNNC1 vector construct
  source 1..2124: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagtagtcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagactccctctctgcgcgctcgctcgctcactgaggccgggcgaccaaaggtcgcccgacgcccgggctttgcccgggcggcctcagtgagcgagcgagcgcgcagagagggagt

SEQ ID NO: 60 - DNA, 3146 bp, synthetic construct
  misc_feature 1..3146: note = Made in Lab - AAV-MHCK7-TNNC1-GFP vector construct
  source 1..3146: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 61 - DNA, 2919 bp, synthetic construct
  misc_feature 1..2919: note = Made in Lab - AAV-hTnT-TNNC1-GFP vector construct
  source 1..2919: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 62 - DNA, 4018 bp, synthetic construct
  misc_feature 1..4018: note = Made in Lab - AAV-CAG-TNNC1-GFP vector construct
  source 1..4018: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 63 - DNA, 2222 bp, synthetic construct
  misc_feature 1..2222: note = Made in Lab - MHCK7-TNNC1 expression cassette construct
  source 1..2222: mol_type = other DNA
cctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagtagtcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaagg

SEQ ID NO: 64 - DNA, 1846 bp, synthetic construct
  misc_feature 1..1846: note = Made in lab - hTnT-TNNC1 expression cassette construct
  source 1..1846: mol_type = other DNA
cctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagtagtcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaagg

SEQ ID NO: 65 - DNA, 1846 bp, synthetic construct; identical to SEQ ID NO: 64
  misc_feature 1..1846: note = Made in lab - hTNT-TNNC1 expression cassette construct
  source 1..1846: mol_type = other DNA

SEQ ID NO: 66 - DNA, 2856 bp, synthetic construct
  misc_feature 1..2856: note = Made in Lab - MHCK7-TNNC1-GFP expression cassette construct
  source 1..2856: mol_type = other DNA
cctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaagg

SEQ ID NO: 67 - DNA, 2629 bp, synthetic construct
  misc_feature 1..2629: note = Made in Lab - hTnT-TNNC1-GFP expression cassette construct
  source 1..2629: mol_type = other DNA
cctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaagg

SEQ ID NO: 68 - DNA, 3728 bp, synthetic construct
  misc_feature 1..3728: note = Made in Lab - CAG-TNNC1-GFP expression cassette construct
  source 1..3728: mol_type = other DNA
cctgaggacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaagg

SEQ ID NO: 69 - protein, 735 aa, unidentified [sequence omitted]

SEQ ID NO: 70 - protein, 736 aa, unidentified
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 71 - protein, 736 aa, unidentified
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 72 - protein, 738 aa, unidentified
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 73 - DNA, 2217 bp, unidentified
  source 1..2217: mol_type = genomic DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 74 - protein, 738 aa, unidentified
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 75 - protein, 601 aa, unidentified
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 76 - protein, 535 aa, unidentified
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 77 - DNA, 133 bp, unidentified; identical to SEQ ID NO: 17
  source 1..133: mol_type = genomic DNA

SEQ ID NO: 78 - protein, 7 aa, synthetic construct
  REGION 1..7: note = Peptide insert
TLAVPFK

SEQ ID NO: 79 - protein, 7 aa, synthetic construct
  REGION 1..7: note = Peptide insert
KFPVALT

SEQ ID NO: 80 - protein, 743 aa, synthetic construct
  REGION 1..743: note = AAV9 variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 81 - DNA, 596 bp, Woodchuck hepatitis virus
  source 1..596: mol_type = genomic DNA
attcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 82 - DNA, 387 bp, Oryctolagus cuniculus
  source 1..387: mol_type = genomic DNA
tggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 83 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaatacaatagcaggcatgctggggatgcggtgggctctatgggtacccaggtgctgaagaattgacccggttcctcctggg

SEQ ID NO: 84 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctggg

SEQ ID NO: 85 - DNA, 225 bp, Bos taurus
  source 1..225: mol_type = genomic DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 86 - DNA, 202 bp, Homo sapiens
  source 1..202: mol_type = genomic DNA
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

(19) United States
(12) Patent Application Publication
(10) Pub. No.: US 2024/0335566 A1
(43) Pub. Date: Oct. 10, 2024

(54) TROPONIN C (TNNC1) GENE THERAPY USING AAV VECTOR

(71) Applicant: Spacecraft Seven, LLC, Cranbury, NJ (US)

(72) Inventors: Christopher Dean HERZOG, Cranbury, NJ (US); Chester Bittencort SACRAMENTO, Cranbury, NJ (US); David RICKS, Cranbury, NJ (US); Raj PRABHAKAR, Cranbury, NJ (US)

(21) Appl. No.: 18/718,023

(22) PCT Filed: Dec. 9, 2022
(86) PCT No.: PCT/US2022/081282

§ 371 Date: Jun. 8, 2024

Related U.S. Application Data
(60) Provisional application No. 63/288,255, filed on Dec. 10, 2021.

(51) Int. Cl. A61K 48/00; A61K 38/17; C07K 14/47; C12N 15/86

(52) U.S. Cl. CPC A61K 48/0058; A61K 38/1709; C07K 14/47; C12N 15/86; C12N 2750/14143; C12N 2830/008; C12N 2830/48; C12N 2830/50

(57) ABSTRACT

Provided herein is a gene therapy for TNNC1 (Troponin C)-related cardiomyopathy, e.g. using an adeno-associated virus (AAV) vector. The promoter of the vector may be a MHCK7 promoter or a cardiac troponin T (hTNNT2) promoter. The capsid may be an AAV9 or AAVrh.74 capsid or a functional variant thereof. Other promoters or capsids may be used. Further provided are methods of treatment, such as by intravenous, intracoronary, intracarotid or intracardiac administration of the rAAV vector, and other compositions and methods.

### CROSS-REFERENCE TO RELATED APPLICATIONS

This application claims benefit of priority to U.S. Provisional Patent Application No. 63/288,255, filed Dec. 10, 2021, the disclosure of which is incorporated herein by reference in its entirety for all purposes.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is ROPA_026_01WO_SeqList_ST26.xml. The text file is about 152,263 bytes, created on Dec. 7, 2022, and is being submitted electronically via EFS-Web.

### BACKGROUND

Mutations in the TNNC1 gene are a major cause of cardiomyopathy. TNNC1, located at 3p21.1, encodes cardiac muscle troponin C, the calcium-binding subunit responsible for sensing myofilament Ca²⁺ and regulating contraction. Troponin C neutralizes the suppression of the contractile interaction between myosin and actin induced by troponin I-tropomyosin. TNNC1 loss of function (LOF) mutations decrease Ca²⁺ sensitivity and binding, leading to dilated cardiomyopathy (DCM). TNNC1 gain of function (GOF) mutations increase Ca²⁺ sensitivity and binding, leading to hypercontractility and hypertrophic cardiomyopathy (HCM).

Clinical manifestations of TNNC1 DCM include heart failure (e.g., a mean ejection fraction (EF) of less than 30%), left ventricular dilation, the need for a heart transplant, and risk of sudden cardiac death. The average age of onset of TNNC1 DCM is about 30 years, but it can present earlier and even in pediatric patients. Clinical manifestations of TNNC1 HCM are dyspnea, syncope, angina, arrhythmia, left ventricular hypertrophy (LVH), and left ventricular outflow tract obstruction (LVOTO).

There remains an unmet need in the art for treatments for TNNC1 DCM, TNNC1 HCM, and other cardiomyopathies associated with mutations in TNNC1. The compositions and methods disclosed herein address this need.

### SUMMARY

The present invention relates generally to gene therapy for a disease or disorder, e.g., a cardiac disease or disorder, using a vector expressing TNNC1 or a functional variant thereof.

Various other aspects and embodiments are disclosed in the detailed description that follows. The invention is limited solely by the appended claims.

### BRIEF DESCRIPTION OF FIGURES

FIG. 1 shows a diagram illustrating a non-limiting example of a vector genome. The full polynucleotide sequence of the vector genome is SEQ ID NO: 57. The underlined portion is the expression cassette (SEQ ID NO: 63).

FIG. 2 shows a diagram illustrating a non-limiting example of a vector genome. The full polynucleotide sequence of the vector genome is SEQ ID NO: 58. The underlined portion is the expression cassette (SEQ ID NO: 64).

FIG. 3 shows a diagram illustrating a non-limiting example of a vector genome. The full polynucleotide sequence of the vector genome is SEQ ID NO: 59. The underlined portion is the expression cassette (SEQ ID NO: 65).

FIG. 4 shows a diagram illustrating a non-limiting example of a vector genome. The full polynucleotide sequence of the vector genome is SEQ ID NO: 60. The underlined portion is the expression cassette (SEQ ID NO: 66).

FIG. 5 shows a diagram illustrating a non-limiting example of a vector genome. The full polynucleotide sequence of the vector genome is SEQ ID NO: 61. The underlined portion is the expression cassette (SEQ ID NO: 67).

FIG. 6 shows a diagram illustrating a non-limiting example of a vector genome. The full polynucleotide sequence of the vector genome is SEQ ID NO: 62. The underlined portion is the expression cassette (SEQ ID NO: 68).

FIG. 7 shows a diagram illustrating a non-limiting example of a vector genome. The full polynucleotide sequence of the vector genome is SEQ ID NO: 57. The MHCK7 promoter as described herein is labelled “Enhancer/MHCK7” in the diagram.

FIG. 8 shows a diagram illustrating a non-limiting example of a vector genome. The full polynucleotide sequence of the vector genome is SEQ ID NO: 58.

FIG. 9 shows a western blot (WB) of human TnC protein expression in transduced CHO-Lec2 cells. The WB shows human TnC (top panel) or loading control, GAPDH (bottom panel). Lane 1 is AAV9-MHCK7-TNNC1 (transduced with MOI of 3E5), lane 2 is AAVrh.74-MHCK7-TNNC1 (transduced with MOI of 3E5), lane 3 is AAV9-hTnT-TNNC1 (transduced with MOI of 3E6), lane 4 is AAVrh.74-hTnT-TNNC1 (transduced with MOI of 3E6) and lane 5 is a control of non-transduced cells.

FIG. 10A and FIG. 10B show Kaplan-Meier survival curves for D73N^{+/-} treated mice (n=7-11 per group). FIG. 10A shows the survival of male treated mice and FIG. 10B shows female survival. All AAV-injected animals lived considerably longer than formulation buffer (FB) injected D73N^{+/-} controls. FB comprises Phosphate Buffered Saline (PBS) with 0.01% Pluronic F-68 and does not include an AAV.

FIG. 11A and FIG. 11B show bar graphs illustrating that significant mitigation of the disease-related increase of End Diastolic Diameter was observed in all male AAV-injected groups, with the greatest effect noted in the AAV9-MHCK7 group compared to formulation buffer (FB)-injected D73N^{+/-} control animals (FIG. 11A). An apparent but nonsignificant effect was observed in female mice, most notably in the AAV9-hTnT-TNNC1 group (FIG. 11B). Statistical analyses (One-way ANOVA) followed by Tukey's multiple comparisons test were performed (*p≤0.05, **p≤0.01, ***p≤0.001, ****p<0.0001).

FIG. 12A and FIG. 12B show bar graphs illustrating significant mitigation of the disease-related increase in End Systolic Diameter (FIG. 12A) in male mice treated with AAV9-MHCK7-TNNC1 and AAVrh.74-TNNC1 compared to FB injected D73N^{+/-} controls was observed. An apparent but nonsignificant effect in female mice, most notably in the AAV9-hTnT-TNNC1 injected group (FIG. 12B). Statistical analyses (One-way ANOVA) followed by Tukey's multiple comparisons test were performed (*p≤0.05, ****p<0.0001).

FIG. 13A and FIG. 13B show bar graphs illustrating significant mitigation of the disease-related progression of dilated cardiomyopathy was revealed by greater Ejection Fraction (%) in AAV9-MHCK7-TNNC1 injected animals compared to FB injected D73N^{+/-} control male mice (FIG. 13A). Statistical analyses (One-way ANOVA) followed by Tukey's multiple comparisons test were performed (*p≤0.05).

FIG. 14A and FIG. 14B show bar graphs illustrating significant mitigation of the normal progression of dilated cardiomyopathy was revealed by greater Fractional Shortening (%) in AAV9-MHCK7-TNNC1 injected animals compared to FB injected D73N^{+/-} control male mice (FIG. 14A). Statistical analyses (One-way ANOVA) followed by Tukey's multiple comparisons test were performed (*p≤0.05).

### DETAILED DESCRIPTION OF THE INVENTION

The present disclosure provided gene therapy vectors for TNNC1 that deliver a polynucleotide encoding TNNC1 or a functional variant thereof, along with methods of use, and other compositions and methods. In some embodiments, the promoter is an MHCK7 promoter. In some embodiments, the AAV vector is an AAV9 vector. In some embodiments, the promoter is an MHCK7 promoter and the AAV vector is an AAV9 vector. In some embodiment, the promoter is an MHCK7 promoter and the AAV vector is a AAVrh.74 vector. In some embodiments, the promoter is a hTNNT2 promoter. In some embodiments, the AAV vector is an AAV9 vector. In some embodiments, the promoter is an hTNNT2 promoter and the AAV vector is an AAV9 vector. In some embodiment, the promoter is an hTNNT2 promoter and the AAV vector is a AAVrh.74 vector.

This disclosure further provides methods of treating a disease or disorder in a subject by administering a gene therapy vector of the disclosure. In a preferred embodiment, the disease or disorder is TNNC1 DCM or TNNC1 HCM.

In accordance with the present invention, a polynucleotide encoding a TNNC1 or functional variant thereof may be employed in generating a gene therapy vector. The resulting vector may be employed in treating diseases or disorders, e.g. TNNC1 DCM, TNNC1 HCM or others.

### Definitions

The section headings are for organizational purposes only and are not to be construed as limiting the subject matter described to particular aspects or embodiments.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

As used throughout the disclosure, sequence “identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to the number or percentage of residues that are the same in a sequence of interest and a reference sequence. The percentage can be calculated by optimally aligning the sequence of interest to the reference sequence; comparing the two sequences over the entire length of the reference sequence; determining the number of positions at which the identical amino acid residue or nucleic acid base occurs in both sequences to yield the number of matched positions; dividing the number of matched positions by the total number of positions in the reference sequence adjusted by adding the number of gap positions introduced into the reference sequence in generating the alignment; and multiplying the result by 100 to yield the percentage of sequence identity. When comparing DNA and RNA, thymine (T) and uracil (U) can be considered equivalent. Identity calculation can be performed manually or by the BLAST algorithm.

As used herein, an “AAV vector” or “rAAV vector” refers to a recombinant vector comprising one or more polynucleotides of interest (or transgenes) that are flanked by AAV terminal repeat sequences (ITRs). Such AAV vectors can be replicated and packaged into infectious viral particles when present in a host cell that has been transfected with a plasmid encoding and expressing rep and cap gene products. Alternatively, AAV vectors can be packaged into infectious particles using a host cell that has been stably engineered to express rep and cap genes.

As used herein, an “AAV virion” or “AAV viral particle” or “AAV vector particle” refers to a viral particle composed of at least one AAV capsid protein and an encapsidated polynucleotide AAV vector. As used herein, if the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

As used herein, “promoter” refers to a polynucleotide sequence capable of promoting initiation of RNA transcription from a polynucleotide in a eukaryotic cell.

As used herein, “vector genome” refers to the polynucleotide sequence packaged by the vector (e.g., an rAAV virion), including flanking sequences (e.g., in AAV, inverted terminal repeats). The terms “expression cassette” and “polynucleotide cassette” refer to the portion of the vector genome between the flanking ITR sequences. “Expression cassette” implies that the vector genome comprises at least one gene encoding a gene product operable linked to an element that drives expression (e.g., a promoter), including any regulatory elements and/or enhancer elements. “Polynucleotide cassette” refers to the portion of the vector genome that comprises at least one gene encoding a gene product operatively linked to an element that drives expression (e.g., a promoter), including any regulatory elements and/or enhancer elements.

As used herein, the term “patient in need” or “subject in need” refers to a patient or subject at risk of, or suffering from, a disease, disorder or condition that is amenable to treatment or amelioration with a recombinant gene therapy vector or gene editing system disclosed herein. A patient or subject in need may, for instance, be a patient or subject diagnosed with a disorder associated with heart. A subject may have a mutation in a TNNC1 gene or deletion of all or a part of the TNNC1 gene, or of gene regulatory sequences, that causes aberrant expression of the TNNC1 protein. “Subject” and “patient” are used interchangeably herein. The subject treated by the methods described herein may be an adult or a child. Subjects may range in age.

As used herein, the term “variant” or “functional variant” refer, interchangeably, to a protein that has one or more amino-acid substitutions, insertions, or deletions compared to a parental protein that retains one or more desired activities of the parental protein.

As used herein, “genetic disruption” refers to a partial or complete loss of function or aberrant activity in a gene. For example, a subject may suffer from a genetic disruption in expression or function in the TNNC1 gene that decreases expression or results in loss or aberrant function of the TNNC1 protein in at least some cells (e.g., cardiac cells) of the subject. “Genetic Disruption” also refers to changes in a gene that lead to gain of function mutations, for example, gain of function mutations in the TNNC1 protein.

As used herein, “treating” refers to ameliorating one or more symptoms of a disease or disorder. The term “preventing” refers to delaying or interrupting the onset of one or more symptoms of a disease or disorder or slowing the progression of TNNC1-related disease or disorder, e.g., TNNC1 DCM and/or TNNC1 HCM.

### TNNC1 Protein or Polynucleotide

The present disclosure contemplates compositions and methods of use related to TNNC1 protein. Various mutations in TNNC1 are known to be associated with TNNC1 DCM or TNNC1 HCM. Examples of mutations associated with TNNC1 DCM or TNNC1 HCM include, without limitation: Y5H, A8V, L29Q, A31S, C84Y, E134D, D132N, D145E, I148V, G159D, G159R, M103I, and/or any combination thereof.

The native sequence of human TNNC1 is shown below. A TNNC1 isoform has a sequence of 161 amino acid residues (GenBank NP_003271.1):

(SEQ ID NO: 1)
MDDIYKAAVEQLTEEQKNEFKAAFDIFVLGAEDGCISTKELGKVMRMLGQ
NPTPEELQEMIDEVDEDGSGTVDFDEFLVMMVRCMKDDSKGKSEEELSDL
FRMFDKNADGYIDLDELKIMLQATGETITEDDIEELMKDGDKNNDGRIDY
DEFLEFMKGVE.

In some embodiments, the TNNC1 protein comprises a polypeptide sequence at least 75%, 80%, 85%, 90%, 92%, 93%, 94%, 95%, 96%, 97%, 98%, 99%, or 100% identical to SEQ ID NO: 1. In some embodiments, the TNNC1 protein is encoded by a polynucleotide that comprises a sequence at least 75%, 80%, 85%, 90%, 92%, 93%, 94%, 95%, 96%, 97%, 98%, 99%, or 100% identical to SEQ ID NO: 2. In some embodiments, the TNNC1 protein has no mutations associated with disease. In some embodiments, the TNNC1 protein is a wild-type or native TNNC1 protein, e.g. human TNNC1.

In some embodiments, the disclosure provides a recombinant adeno-associated virus (rAAV) virion, comprising a capsid and a vector genome, wherein the vector genome comprises a polynucleotide sequence encoding an TNNC1 or a functional variant thereof, operatively linked to a promoter. In some embodiments, the disclosure provides a recombinant adeno-associated virus (rAAV) virion, comprising a capsid and a vector genome, wherein the vector genome comprises a polynucleotide sequence encoding an TNNC1, operatively linked to a promoter. In some embodiments, the TNNC1 protein comprises a polypeptide sequence at least 75%, 80%, 85%, 90%, 92%, 93%, 94%, 95%, 96%, 97%, 98%, 99%, or 100% identical to SEQ ID NO: 1. The polynucleotide encoding the TNNC1 may comprise a polynucleotide sequence at least 75%, 80%, 85%, 90%, 92%, 93%, 94%, 95%, 96%, 97%, 98%, 99%, or 100% identical to:

(SEQ ID NO: 2)
atggatgacatctacaaggctgcggtagagcagctgacagaagagcagaa
aaatgagttcaaggcagccttcgacatcttcgtgctgggcgctgaggatg
gctgcatcagcaccaaggagctgggcaaggtgatgaggatgctgggccag
aaccccacccctgaggagctgcaggagatgatcgatgaggtggacgagga
cggcagcggcacggtggactttgatgagttcctggtcatgatggttcggt
gcatgaaggacgacagcaaagggaaatctgaggaggagctgtctgacctc
ttccgcatgtttgacaaaaatgctgatggctacatcgacctggatgagct
gaagataatgctgcaggctacaggcgagaccatcacggaggacgacatcg
aggagctcatgaaggacggagacaagaacaacgacggccgcatcgactat
gatgagttcctggagttcatgaagggtgtggag.

Optionally, the polynucleotide sequence encoding the vector genome may comprise a Kozak sequence, including but not limited to GCCACCATGG (SEQ ID NO: 3). Kozak sequence may overlap the polynucleotide sequence encoding an TNNC1 protein or a functional variant thereof. For example, the vector genome may comprise a polynucleotide sequence (with Kozak underlined) at least 75%, 80%, 85%, 90%, 92%, 93%, 94%, 95%, 96%, 97%, 98%, 99%, or 100% identical to:

(SEQ ID NO: 4)
GCCACCATGGatgacatctacaaggctgcggtagagcagctgacagaaga
gcagaaaaatgagttcaaggcagccttcgacatcttcgtgctgggcgctg
aggatggctgcatcagcaccaaggagctgggcaaggtgatgaggatgctg
ggccagaaccccacccctgaggagctgcaggagatgatcgatgaggtgga
cgaggacggcagcggcacggtggactttgatgagttcctggtcatgatgg
ttcggtgcatgaaggacgacagcaaagggaaatctgaggaggagctgtct
gacctcttccgcatgtttgacaaaaatgctgatggctacatcgacctgga
tgagctgaagataatgctgcaggctacaggcgagaccatcacggaggacg
acatcgaggagctcatgaaggacggagacaagaacaacgacggccgcatc
gactatgatgagttcctggagttcatgaagggtgtggag.

In some embodiments, the Kozak sequence is an alternative Kozak sequence comprising or consisting of any one of:

| (SEQ ID NO: 5) | |
|---|---|
| | (gcc)gccRccAUGG; |
| (SEQ ID NO: 6) | |
| | AGNNAUGN; |
| (SEQ ID NO: 7) | |
| | ANNAUGG; |
| (SEQ ID NO: 8) | |
| | ANNAUGC; |
| (SEQ ID NO: 9) | |
| | ACCAUGG; |
| | and |
| (SEQ ID NO: 10) | |
| | GACACCAUGG |

In some embodiments, the vector genome comprises no Kozak sequence. The polynucleotide sequence may be codon-optimized.

### Vector Genome

The AAV virions of the disclosure comprise a vector genome. The vector genome may comprise an expression cassette (or a polynucleotide cassette for gene-editing applications not requiring expression of the polynucleotide sequence). Any suitable inverted terminal repeats (ITRs) may be used. The ITRs may be from the same serotype as the capsid or a different serotype (e.g., AAV2 ITRs may be used).

In some embodiments, the 5′ ITR comprises an AAV ITR. In some embodiments, the 5′ ITR comprises a polynucleotide sequence at least 75%, 80%, 85%, 90%, 92%, 93%, 94%, 95%, 96%, 97%, 98%, 99%, or 100% identical to:

(SEQ ID NO: 11)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCT.

In some embodiments, the 5′ ITR comprises an AAV2 ITR. In some embodiments, the 5′ ITR comprises a polynucleotide sequence at least 75%, 80%, 85%, 90%, 92%, 93%, 94%, 95%, 96%, 97%, 98%, 99%, or 100% identical to:

(SEQ ID NO: 12)
GCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGA
GTGGCCAACTCCATCACTAGGGGTTCCTTGTAGTTAATGATTAACCCGCC
ATGCTACTTATCTACGTA.

In some embodiments, the 5′ ITR comprises an AAV ITR. In some embodiments, the 5′ ITR comprises a polynucleotide sequence at least 75%, 80%, 85%, 90%, 92%, 93%, 94%, 95%, 96%, 97%, 98%, 99%, or 100% identical to:

(SEQ ID NO: 13)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACGTA.

In some embodiments, the 5′ ITR comprises an AAV ITR. In some embodiments, the 5′ ITR comprises a polynucleotide sequence at least 75%, 80%, 85%, 90%, 92%, 93%, 94%, 95%, 96%, 97%, 98%, 99%, or 100% identical to:

(SEQ ID NO: 14)
TTGGCCACTCCCTCTCTGCGCGCTCGCTCGCTCACTGAGGCCGCCCGGGC
AAAGCCCGGGCGTCGGGCGACCTTTGGTCGCCCGGCCTCAGTGAGCGAGC
GAGCGCGCAGAGAGGGAGTGGCCAACTCCATCACTAGGGGTTCCT.

In some embodiments, the 3′ ITR comprises an AAV ITR. In some embodiments, the 5′ ITR comprises a polynucleotide sequence at least 75%, 80%, 85%, 90%, 92%, 93%, 94%, 95%, 96%, 97%, 98%, 99%, or 100% identical to:

(SEQ ID NO: 15)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG.

In some embodiments, the 3′ ITR comprises an AAV2 ITR. In some embodiments, the 5′ ITR comprises a polynucleotide sequence at least 75%, 80%, 85%, 90%, 92%, 93%, 94%, 95%, 96%, 97%, 98%, 99%, or 100% identical to:

(SEQ ID NO: 16)
TACGTAGATAAGTAGCATGGCGGGTTAATCATTAACTACAAGGAACCCCT
AGTGATGGAGTTGGCCACTCCCTCTCTGCGCGCTCGCTCGCTCACTGAGG
CCGGGCGACCAAAGGTCGCCCGACGCCCGGGCTTTGCCCGGGCGGCCTCA
GTGAGCGAGCGAGCGCGC.

In some embodiments, the 3′ ITR comprises an AAV2 ITR. In some embodiments, the 5′ ITR comprises a polynucleotide sequence at least 75%, 80%, 85%, 90%, 92%, 93%, 94%, 95%, 96%, 97%, 98%, 99%, or 100% identical to:

(SEQ ID NO: 77)
AGGAACCCCTAGTGATGGAGACTCCCTCTCTGCGCGCTCGCTCGCTCACT
GAGGCCGGGCGACCAAAGGTCGCCCGACGCCCGGGCTTTGCCCGGGCGGC
CTCAGTGAGCGAGCGAGCGCGCAGAGAGGGAGT.

In some embodiments, the 3′ ITR comprises an AAV2 ITR. In some embodiments, the 5′ ITR comprises a polynucleotide sequence at least 75%, 80%, 85%, 90%, 92%, 93%, 94%, 95%, 96%, 97%, 98%, 99%, or 100% identical to:

(SEQ ID NO: 17)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGAGAGGGAGTGGCCAA.

In some embodiments the vector genome comprises one or more filler sequences, e.g., at least 75%, 80%, 85%, 90%, 92%, 93%, 94%, 95%, 96%, 97%, 98%, 99%, or 100% identical to:

(SEQ ID NO: 18)
GCGGCAATTCAGTCGATAACTATAACGGTCCTAAGGTAGCGATTTAAATA
CGCGCTCTCTTAAGGTAGCCCCGGGACGCGTCAATTGACTACAAACCGAG
TATCTGCAGAGGGCCCTGCGTATG;
(SEQ ID NO: 19)
CTTCTGAGGCGGAAAGAACCAGATCCTCTCTTAAGGTAGCATCGAGATTT
AAATTAGGGATAACAGGGTAATGGCGCGGGCCGC;
or
(SEQ ID NO: 20)
GTTACCCAGGCTGGAGTGCAGTGGCACATTTCTGCTCACTGCAACCTCCT
CCTCCCTGGGTTC.

### Promoters

In some embodiments, the polynucleotide sequence encoding an TNNC1 protein or functional variant thereof is operably linked to a promoter. In preferred embodiments, the promoter is an MHCK7 promoter, which includes enhancer/promoter regions of murine muscle creatine kinase (MCK) and enhancer region of α-myosin heavy-chain genes. (Salva M Z et al., Mol. Ther. 15(2):320-9 (2007).)

The present disclosure contemplates use of various promoters. Promoters useful in embodiments of the present disclosure include, without limitation, a cytomegalovirus (CMV) promoter, phosphoglycerate kinase (PGK) promoter, or a promoter sequence comprised of the CMV enhancer and portions of the chicken beta-actin promoter and the rabbit beta-globin gene (CAG). In some cases, the promoter may be a synthetic promoter. Exemplary synthetic promoters are provided by Schlabach et al. PNAS USA. 107(6):2538-43 (2010).

In some embodiments, a polynucleotide sequence encoding an TNNC1 protein or functional variant thereof is operatively linked to an inducible promoter. An inducible promoter may be configured to cause the polynucleotide sequence to be transcriptionally expressed or not transcriptionally expressed in response to addition or accumulation of an agent or in response to removal, degradation, or dilution of an agent. The agent may be a drug. The agent may be tetracycline or one of its derivatives, including, without limitation, doxycycline. In some cases, the inducible promoter is a tet-on promoter, a tet-off promoter, a chemically-regulated promoter, a physically-regulated promoter (i.e., a promoter that responds to presence or absence of light or to low or high temperature). Inducible promoters include heavy metal ion inducible promoters (such as the mouse mammary tumor virus (mMTV) promoter or various growth hormone promoters), and the promoters from T7 phage which are active in the presence of T7 RNA polymerase. This list of inducible promoters is non-limiting.

In some cases, the promoter is a tissue-specific promoter, such as a promoter capable of driving expression in a cardiac cell to a greater extent than in a non-cardiac cell. In some embodiments, tissue-specific promoter is a selected from any various cardiac cell-specific promoters including but not limited to, desmin (Des), alpha-myosin heavy chain (α-MHC), myosin light chain 2 (MLC-2), cardiac troponin C (cTnC), cardiac troponin T (hTNNT2), muscle creatine kinase (CK) and combinations of promoter/enhancer regions thereof, such as MHCK7. In some cases, the promoter is a ubiquitous promoter. A “ubiquitous promoter” refers to a promoter that is not tissue-specific under experimental or clinical conditions. In some cases, the ubiquitous promoter is any one of CMV, CAG, UBC, PGK, EF1-alpha, GAPDH, SV40, HBV, chicken beta-actin, and human beta-actin promoters.

In some embodiments, the promoter sequence is selected from Table 3. In some embodiments, the promoter comprises a polynucleotide sequence at least 75%, 80%, 85%, 90%, 92%, 93%, 94%, 95%, 96%, 97%, 98%, 99%, or 100% identical to any one of SEQ ID NOS 21-35.

TABLE 3

| | | SEQ ID |
|---|---|---|
| PROMOTER | SEQUENCE | NO: |
| MHCK7 | ACCCTTCAGATTAAAAATAACTGAGGTAAGGGCCTGGG | 21 |
| | TAGGGGAGGTGGTGTGAGACGCTCCTGTCTCTCCTCTA | |
| | TCTGCCCATCGGCCCTTTGGGGAGGAGGAATGTGCCCA | |
| | AGGACTAAAAAAAGGCCATGGAGCCAGAGGGGCGAGGG | |
| | CAACAGACCTTTCATGGGCAAACCTTGGGGCCCTGCTG | |
| | TCTAGCATGCCCCACTACGGGTCTAGGCTGCCCATGTA | |
| | AGGAGGCAAGGCCTGGGGACACCCGAGATGCCTGGTTA | |
| | TAATTAACCCAGACATGTGGCTGCCCCCCCCCCCCCAA | |
| | CACCTGCTGCCTCTAAAAATAACCCTGTCCCTGGTGGA | |
| | TCCCCTGCATGCGAAGATCTTCGAACAAGGCTGTGGGG | |
| | GACTGAGGGCAGGCTGTAACAGGCTTGGGGGCCAGGGC | |
| | TTATACGTGCCTGGGACTCCCAAAGTATTACTGTTCCA | |
| | TGTTCCCGGCGAAGGGCCAGCTGTCCCCCGCCAGCTAG | |
| | ACTCAGCACTTAGTTTAGGAACCAGTGAGCAAGTCAGC | |
| | CCTTGGGGCAGCCCATACAAGGCCATGGGGCTGGGCAA | |
| | GCTGCACGCCTGGGTCCGGGGTGGGCACGGTGCCCGGG | |
| | CAACGAGCTGAAAGCTCATCTGCTCTCAGGGGCCCCTC | |
| | CCTGGGGACAGCCCCTCCTGGCTAGTCACACCCTGTAG | |
| | GCTCCTCTATATAACCCAGGGGCACAGGGGCTGCCCTC | |
| | ATTCTACCACCACCTCCACAGCACAGACAGACACTCAG | |
| | GAGCCAGCCAG | |
| Human cardiac | CTCAGTCCATTAGGAGCCAGTAGCCTGGAAGATGTCTT | 22 |
| troponin T | TACCCCCAGCATCAGTTCAAGTGGAGCAGCACATAACT | |
| promoter | CTTGCCCTCTGCCTTCCAAGATTCTGGTGCTGAGACTT | |
| (without | ATGGAGTGTCTTGGAGGTTGCCTTCTGCCCCCCAACCC | |
| exon 1) | TGCTCCCAGCTGGCCCTCCCAGGCCTGGGTTGCTGGCC | |
| hTnnT2/ | TCTGCTTTATCAGGATTCTCAAGAGGGACAGCTGGTTT | |
| HTNNT2 | ATGTTGCATGACTGTTCCCTGCATATCTGCTCTGGTTT | |
| | TAAATAGCTTATCTGAGCAGCTGGAGGACCACATGGGC | |
| | TTATATGGCGTGGGGTACATGTTCCTGTAGCCTTGTCC | |
| | CTGGCACCTGCCAAAATAGCAGCCAACACCCCCCACCC | |
| | CCACCGCCATCCCCCTGCCCCACCCGTCCCCTGTCGCA | |
| | CATTCCTCCCTCCGCAGGGCTGGCTCACCAGGCCCCAG | |
| | CCCACATGCCTGCTTAAAGCCCTCTCCATCCTCTGCCT | |
| | CACCCAGT | |
| Human cardiac | CTCAGTCCATTAGGAGCCAGTAGCCTGGAAGATGTCTT | 23 |
| troponin T | TACCCCCAGCATCAGTTCAAGTGGAGCAGCACATAACT | |
| promoter (with | CTTGCCCTCTGCCTTCCAAGATTCTGGTGCTGAGACTT | |
| exon 1, | ATGGAGTGTCTTGGAGGTTGCCTTCTGCCCCCCAACCC | |
| underlined) | TGCTCCCAGCTGGCCCTCCCAGGCCTGGGTTGCTGGCC | |
| hTnnT2/ | TCTGCTTTATCAGGATTCTCAAGAGGGACAGCTGGTTT | |
| HTNNT2 | ATGTTGCATGACTGTTCCCTGCATATCTGCTCTGGTTT | |
| | TAAATAGCTTATCTGAGCAGCTGGAGGACCACATGGGC | |
| | TTATATGGCGTGGGGTACATGTTCCTGTAGCCTTGTCC | |
| | CTGGCACCTGCCAAAATAGCAGCCAACACCCCCCACCC | |
| | CCACCGCCATCCCCCTGCCCCACCCGTCCCCTGTCGCA | |
| | CATTCCTCCCTCCGCAGGGCTGGCTCACCAGGCCCCAG | |
| | CCCACATGCCTGCTTAAAGCCCTCTCCATCCTCTGCCT | |
| | CACCCAGTCCCCGCTGAGACTGAGCAGACGCCTCCAGG | |
| | ATCTGTCGGCAG | |
| Mouse a- | GGTACCGGATCCTGCAAGGTCACACAAGGGTCTCCACC | 24 |
| cardiac myosin | CACCAGGTGCCCTAGTCTCAATTTCAGTTTCCATGCCT | |
| heavy chain | TGTTCTCACAATGCTGGCCTCCCCAGAGCTAATTTGGA | |
| promoter | CTTTGTTTTTATTTCAAAAGGGCCTGAATGAGGAGTAG | |
| (aMHC) | ATCTTGTGCTACCCAGCTCTAAGGGTGCCCGTGAAGCC | |
| | CTCAGACCTGGAGCCTTTGCAACAGCCCTTTAGGTGGA | |
| | AGCAGAATAAAGCAATTTTCCTTAAAGCCAAAATCCTG | |
| | CCTCTAGACTCTTCTTCTCTGACCTCGGTCCCTGGGCT | |
| | CTAGGGTGGGGAGGTGGGGCTTGGAAGAAGAAGGTGGG | |
| | GAAGTGGCAAAAGCCGATCCCTAGGGCCCTGTGAAGTT | |
| | CGGAGCCTTCCCTGTACAGCACTGGCTCATAGATCCTC | |
| | CTCCAGCCAAACATAGCAAGAAGTGATACCTCCTTTGT | |
| | GACTTCCCCAGGCCCAGTACCTGTCAGGTTGAAACAGG | |
| | ATTTAGAGAAGCCTCTGAACTCACCTGAACTCTGAAGC | |
| | TCATCCACCAAGCAAGCACCTAGGTGCCACTGCTAGTT | |
| | AGTATCCTACGCTGATAATATGCAGAGCTGGGCCACAG | |
| | AAGTCCTGGGGTGTAGGAACTGACCAGTGACTTTTCAG | |
| | TCGGCAAAGGTATGACCCCCTCAGCAGATGTAGTAATG | |
| | TCCCCTTAGATCCCATCCCAGGCAGGTCTCTAAGAGGA | |
| | CATGGGATGAGAGATGTAGTCATGTGGCATTCCAAACA | |
| | CAGCTATCCACAGTGTCCCTTGCCCCTTCCACTTAGCC | |
| | AGGAGGACAGTAACCTTAGCCTATCTTTCTTCCTCCCC | |
| | ATCCTCCCAGGACACACCCCCTGGTCTGCAGTATTCAT | |
| | TTCTTCCTTCACGTCCCCTCTGTGACTTCCATTTGCAA | |
| | GGCTTTTGACCTCTGCAGCTGCTGGAAGATAGAGTTTG | |
| | GCCCTAGGTGTGGCAAGCCATCTCAAGAGAAAGCAGAC | |
| | AACAGGGGGACCAGATTTTGGAAGGATCAGGAACTAAA | |
| | TCACTGGCGGGCCTGGGGGTAGAAAAAAGAGTGAGTGA | |
| | GTCCGCTCCAGCTAAGCCAAGCTAGTCCCCGAGATACT | |
| | CTGCCACAGCTGGGCTGCTCGGGGTAGCTTTAGGAATG | |
| | TGGGTCTGAAAGACAATGGGATTGGAAGACATCTCTTT | |
| | GAGTCTCCCCTCAACCCCACCTACAGACACACTCGTGT | |
| | GTGGCCAGACTCCTGTTCAACAGCCCTCTGTGTTCTGA | |
| | CCACTGAGCTAGGCAACCAGAGCATGGGCCCTGTGCTG | |
| | AGGATGAAGAGTTGGTTACCAATAGCAAAAACAGCAGG | |
| | GGAGGGAGAACAGAGAACGAAATAAGGAAGGAAGAAGG | |
| | AAAGGCCAGTCAATCAGATGCAGTCAGAAGAGATGGGA | |
| | AGCCAACACACAGCTTGAGCAGAGGAAACAGAAAAGGG | |
| | AGAGATTCTGGGCATAAGGAGGCCACAGAAAGAAGAGC | |
| | CCAGGCCCCCCAAGTCTCCTCTTTATACCCTCATCCCG | |
| | TCTCCCAATTAAGCCCACTCTTCTTCCTAGATCAGACC | |
| | TGAGCTGCAGCGAAGAGACCCGTAGGGAGGATCACACT | |
| | GGATGAAGGAGATGTGTGGAGAAGTCCAGGGAACCTAA | |
| | GAGCCAGAGCCTAAAAGAGCAAGAGATAAAGGTGCTTC | |
| | AAAGGTGGCCAGGCTGTGCACACAGAGGGTCGAGGACT | |
| | GGTGGTAGAGCCTCAAGATAAGGATGATGCTCAGAATG | |
| | GGCGGGGGGGGGGATTCTGGGGGGGGGAGAGAGAAGGT | |
| | GAGAAGGAGCCTGGAACAGAGAATCTGGAAGCGCTGGA | |
| | AACGATACCATAAAGGGAAGAACCCAGGCTACCTTTAG | |
| | ATGTAAATCATGAAAGACAGGGAGAAGGGAAGCTGGAG | |
| | AGAGTAGAAGGACCCCGGGGCAAGACATTGAAGCAAGG | |
| | ACAAGCCAGGTTGAGCGCTCCGTGAAATCAGCCTGCTG | |
| | AAGGCAGAGCCCTGGTATGAGCACCAGAACAGCAGAGG | |
| | CTAGGGTTAATGTCGAGACAGGGAACAGAAGGTAGACA | |
| | CAGGAACAGACAGAGACGGGGGAGCCAGGTAACAAAGG | |
| | AATGGTCCTTCTCACCTGTGGCCAGAGCGTCCATCTGT | |
| | GTCCACATACTCTAGAATGTTCATCAGACTGCAGGGCT | |
| | GGCTTGGGAGGCAGCTGGAAAGAGTATGTGAGAGCCAG | |
| | GGGAGACAAGGGGGCCTAGGAAAGGAAGAAGAGGGCAA | |
| | ACCAGGCCACACAAGAGGGCAGAGCCCAGAACTGAGTT | |
| | AACTCCTTCCTTGTTGCATCTTCCATAGGAGGCAGTGG | |
| | GAACTCTGTGACCACCATCCCCCATGAGCCCCCACTAC | |
| | CCATACCAAGTTTGGCCTGAGTGGCATTCTAGGTTCCC | |
| | TGAGGACAGAGCCTGGCCTTTGTCTCTTGGACCTGACC | |
| | CAAGCTGACCCAATGTTCTCAGTACCTTATCATGCCCT | |
| | CAAGAGCTTGAGAACCAGGCAGTGACATATTAGGCCAT | |
| | GGGCTAACCCTGGAGCTTGCACACAGGAGCCTCAAGTG | |
| | ACCTCCAGGGACACAGCTGCAGACAGGTGGCCTTTATC | |
| | CCCAAAGAGCAACCATTTGGCATAGGTGGCTGCAAATG | |
| | GGAATGCAAGGTTGAATCAGGTCCCTTCAAGAATACTG | |
| | CATGCAAGACCTAAGACCCCTGGAGAGAGGGGTATGCT | |
| | CCTGCCCCCACCCACCATAAGGGGAGTGAACTATCCTA | |
| | GGGGGCTGGCGACCTTGGGGAGACACCACATTACTGAG | |
| | AGTGCTGAGCCCAGAAAAACTGACCGCCCTGTGTCCTG | |
| | CCCACCTCCACACTCTAGAGCTATATTGAGAGGTGACA | |
| | GTAGATAGGGTGGGAGCTGGTAGCAGGGAGAGTGTTCC | |
| | TGGGTGTGAGGGTGTAGGGGAAAGCCAGAGCAGGGGAG | |
| | TCTGGCTTTGTCTCCTGAACACAATGTCTACTTAGTTA | |
| | TAACAGGCATGACCTGCTAAAGACCCAACATCTACGAC | |
| | CTCTGAAAAGACAGCAGCCCTGGAGGACAGGGGTTGTC | |
| | TCTGAGCCTTGGGTGCTTGATGGTGCCACAAAGGAGGG | |
| | CATGAGTGTGAGTATAAGGCCCCAGGAGCGTTAGAGAA | |
| | GGGCACTTGGGAAGGGGTCAGTCTGCAGAGCCCCTATC | |
| | CATGGAATCTGGAGCCTGGGGCCAACTGGTGTAAATCT | |
| | CTGGGCCTGCCAGGCATTCAAAGCAGCACCTGCATCCT | |
| | CTGGCAGCCTGGGGAGGCGGAAGGGAGCAACCCCCCAC | |
| | TTATACCCTTTCTCCCTCAGCCCCAGGATTAACACCTC | |
| | TGGCCTTCCCCCTTCCCACCTCCCATCAGGAGTGGAGG | |
| | GTTGCAGAGGGAGGGTAAAAACCTACATGTCCAAACAT | |
| | CATGGTGCACGATATATGGATCAGTATGTGTAGAGGCA | |
| | AGAAAGGAAATCTGCAGGCTTAACTGGGTTAATGTGTA | |
| | AAGTCTGTGTGCATGTGTGTGTGTCTGACTGAAAACGG | |
| | GCATGGCTGTGCAGCTGTTCAGTTCTGTGCGTGAGGTT | |
| | ACCAGACTGCAGGTTTGTGTGTAAATTGCCCAAGGCAA | |
| | AGTGGGTGAATCCCTTCCATGGTTTAAAGAGATTGGAT | |
| | GATGGCCTGCATCTCAAGGACCATGGAAAATAGAATGG | |
| | ACACTCTATATGTGTCTCTAAGCTAAGGTAGCAAGGTC | |
| | TTTGGAGGACACCTGTCTAGAGATGTGGGCAACAGAGA | |
| | CTACAGACAGTATCTGTACAGAGTAAGGAGAGAGAGGA | |
| | GGGGGTGTAGAATTCTCTTACTATCAAAGGGAAACTGA | |
| | GTCGTGCACCTGCAAAGTGGATGCTCTCCCTAGACATC | |
| | ATGACTTTGTCTCTGGGGAGCCAGCACTGTGGAACTTC | |
| | AGGTCTGAGAGAGTAGGAGGCTCCCCTCAGCCTGAAGC | |
| | TATGCAGATAGCCAGGGTTGAAAGGGGGAAGGGAGAGC | |
| | CTGGGATGGGAGCTTGTGTGTTGGAGGCAGGGGACAGA | |
| | TATTAAGCCTGGAAGAGAAGGTGACCCTTACCCAGTTG | |
| | TTCAACTCACCCTTCAGATTAAAAATAACTGAGGTAAG | |
| | GGCCTGGGTAGGGGAGGTGGTGTGAGACGCTCCTGTCT | |
| | CTCCTCTATCTGCCCATCGGCCCTTTGGGGAGGAGGAA | |
| | TGTGCCCAAGGACTAAAAAAAGGCCATGGAGCCAGAGG | |
| | GGCGAGGGCAACAGACCTTTCATGGGCAAACCTTGGGG | |
| | CCCTGCTGTCCTCCTGTCACCTCCAGAGCCAAGGGATC | |
| | AAAGGAGGAGGAGCCAGGACAGGAGGGAAGTGGGAGGG | |
| | AGGGTCCCAGCAGAGGACTCCAAATTTAGGCAGCAGGC | |
| | ATATGGGATGGGATATAAAGGGGCTGGAGCACTGAGAG | |
| | CTGTCAGAGATTTCTCCAACCCAGGTAAGAGGGAGTTT | |
| | CGGGTGGGGGCTCTTCACCCACACCAGACCTCTCCCCA | |
| | CCTAGAAGGAAACTGCCTTTCCTGGAAGTGGGGTTCAG | |
| | GCCGGTCAGAGATCTGACAGGGTGGCCTTCCACCAGCC | |
| | TGGGAAGTTCTCAGTGGCAGGAGGTTTCCACAAGAAAC | |
| | ACTGGATGCCCCTTCCCTTACGCTGTCTTCTCCATCTT | |
| | CCTCCTGGGGATGCTCCTCCCCGTCTTGGTTTATCTTG | |
| | GCTCTTCGTCTTCAGCAAGATTTGCCCTGTGCTGTCCA | |
| | CTCCATCTTTCTCTACTGTCTCCGTGCCTTGCCTTGCC | |
| | TTCTTGCGTGTCCTTCCTTTCCACCCATTTCTCACTTC | |
| | ACCTTTTCTCCCCTTCTCATTTGTATTCATCCTTCCTT | |
| | CCTTCCTTCCTTCCTTCCTTCCTTCCTTCCTTCCTTCC | |
| | TTTCTCCCTTCCTTCCTTCCTTCCTTCCTTCCTTCCTT | |
| | CCTTCCTTCCTGTGTCAGAGTGCTGAGAATCACACCTG | |
| | GGGTTCCCACCCTTATGTAAACAATCTTCCAGTGAGCC | |
| | ACAGCTTCAGTGCTGCTGGGTGCTCTCTTACCTTCCTC | |
| | ACCCCCTGGCTTGTCCTGTTCCATCCTGGTCAGGATCT | |
| | CTAGATTGGTCTCCCAGCCTCTGCTACTCCTCTTCCTG | |
| | CCTGTTCCTCTCTCTGTCCAGCTGCGCCACTGTGGTGC | |
| | CTCGTTCCAGCTGTGGTCCACATTCTTCAGGATTCTCT | |
| | GAAAAGTTAACCAGGTGAGAATGTTTCCCCTGTAGACA | |
| | GCAGATCACGATTCTCCCGGAAGTCAGGCTTCCAGCCC | |
| | TCTCTTTCTCTGCCCAGCTGCCCGGCACTCTTAGCAAA | |
| | CCTCAGGCACCCTTACCCCACATAGACCTCTGACAGAG | |
| | AAGCAGGCACTTTACATGGAGTCCTGGTGGGAGAGCCA | |
| | TAGGCTACGGTGTAAAAGAGGCAGGGAAGTGGTGGTGT | |
| | AGGAAAGTCAGGACTTCACATAGAAGCCTAGCCCACAC | |
| | CAGAAATGACAGACAGATCCCTCCTATCTCCCCCATAA | |
| | GAGTTTGAGTCGACCCGCGGCCCCGAATTG | |
| Chicken | GGGATAAAAGCAGTCTGGGCTTTCACATGACAGCATCT | 25 |
| cardiac | GGGGCTGCGGCAGAGGGTCGGGTCCGAAGCGCTGCCTT | |
| troponin T | ATCAGCGTCCCCAGCCCTGGGAGGTGACAGCTGGCTGG | |
| promoter | CTTGTGTCAGCCCCTCGGGCACTCACGTATCTCCGTCC | |
| (cTnT) | GACGGGTTTAAAATAGCAAAACTCTGAGGCCACACAAT | |
| | AGCTTGGGCTTATATGGGCTCCTGTGGGGGAAGGGGGA | |
| | GCACGGAGGGGGCCGGGGCCGCTGCTGCCAAAATAGCA | |
| | GCTCACAAGTGTTGCATTCCTCTCTGGGCGCCGGGCAC | |
| | ATTCCTGCTGGCTCTGCCCGCCCCGGGGTGGGCGCCGG | |
| | GGGGACCTTAAAGCCTCTGCCCCCCAAGGAGCCCTTCC | |
| | CAGACAGCCGCCGGCACCCACCGCTCCGTGGGA | |
| Human | CTCTCAGCCCTGGAAGTCCTTGCTCACAGCCGAGGCGC | 26 |
| Creatine | CGAGAGCGCTTGCTCTGCCCAGATCTGCGCGAGTCTGG | |
| Kinase M | CGCCCGCGCTCTGAACGGCGTCGCTGCCCAGCCCCCTT | |
| (hCKM) | CCCCGGGAGGTGGGAGCGGCCACCCAGGGCCCCGTGGC | |
| | TGCCCTTGTAAGGAGGCGAGGCCCGAGGACACCCGAGA | |
| | CGCCCGGTTATAATTAACCAGGACACGTGGCGAACCCC | |
| | CCTCCAACACCTGCCCCCGAACCCCCCCATACCCAGCG | |
| | CCTCGGGTCTCGGCCTTTGCGGCAGAGGAGACAGCAAA | |
| | GCGCCCTCTAAAAATAACTCCTTTCCCGGCGACCGAGA | |
| | CCCTCCCTGTCCCCCGCACAGCGGAAATCTCCCAGTGG | |
| | CACCGAGGGGGCGAGGGTTAAGTGGGGGGGAGGGTGAC | |
| | CACCGCCTCCCACCCTTGCCCTGAGTTTGAATCTCTCC | |
| | AACTCAGCCAGCCTCAGTTTCCCCTCCACTCAGTCCCT | |
| | AGGAGGAAGGGGCGCCCAAGCGCGGGTTTCTGGGGTTA | |
| | GACTGCCCTCCATTGCAATTGGTCCTTCTCCCGGCCTC | |
| | TGCTTCCTCCAGCTCACAGGGTATCTGCTCCTCCTGGA | |
| | GCCACACCTTGGTTCCCCGAGGTGCCGCTGGGACTCGG | |
| | GTAGGGGTGAGGGCCCAGGGGGCACAGGGGGAGCCGAG | |
| | GGCCACAGGAAGGGCTGGTGGCTGAAGGAGACTCAGGG | |
| | GCCAGGGGACGGTGGCTTCTACGTGCTTGGGACGTTCC | |
| | CAGCCACCGTCCCATGTTCCCGGCGGGGGGCCAGCTGT | |
| | CCCCACCGCCAGCCCAACTCAGCACTTGGTCAGGGTAT | |
| | CAGCTTGGTGGGGGGGCGTGAGCCCAGCCCCTGGGGCG | |
| | GCTCAGCCCATACAAGGCCATGGGGCTGGGCGCAAAGC | |
| | ATGCCTGGGTTCAGGGTGGGTATGGTGCGGGAGCAGGG | |
| | AGGTGAGAGGCTCAGCTGCCCTCCAGAACTCCTCCCTG | |
| | GGGACAACCCCTCCCAGCCAATAGCACAGCCTAGGTCC | |
| | CCCTATATAAGGCCACGGCTGCTGGCCCTTCCTTTGGG | |
| | TCAGTGTCACCTCCAGGATACAGACA | |
| Human beta- | GCCCAGCACCCCAAGGCGGCCAACGCCAAAACTCTCCC | 27 |
| actin (HuBa) | TCCTCCTCTTCCTCAATCTCGCTCTCGCTCTTTTTTTT | |
| | TTTCGCAAAAGGAGGGGAGAGGGGGTAAAAAAATGCTG | |
| | CACTGTGCGGCGAAGCCGGTGAGTGAGCGGCGCGGGGC | |
| | CAATCAGCGTGCGCCGTTCCGAAAGTTGCCTTTTATGG | |
| | CTCGAGCGGCCGCGGCGGCGCCCTATAAAACCCAGCGG | |
| | CGCGACGCGCCACCACCGCCGAGTC | |
| Chicken beta- | GGTCGAGGTGAGCCCCACGTTCTGCTTCACTCTCCCCA | 28 |
| actin (CBA) | TCTCCCCCCCCTCCCCACCCCCAATTTTGTATTTATTT | |
| | ATTTTTTAATTATTTTGTGCAGCGATGGGGGCGGGGGG | |
| | GGGGGGGGCGCGCGCCAGGCGGGGCGGGGCGGGGCGAG | |
| | GGGCGGGGCGGGGCGAGGCGGAGAGGTGCGGCGGCAGC | |
| | CAATCAGAGCGGCGCGCTCCGAAAGTTTCCTTTTATGG | |
| | CGAGGCGGCGGCGGCGGCGGCCCTATAAAAAGCGAAGC | |
| | GCGCGGCGGGCGGGA | |
| Cytomegalo- | TGGTGATGCGGTTTTGGCAGTACACCAATGGGCGTGGA | 29 |
| virus (CMV) | TAGCGGTTTGACTCACGGGGATTTCCAAGTCTCCACCC | |
| | CATTGACGTCAATGGGAGTTTGTTTTGGCACCAAAATC | |
| | AACGGGACTTTCCAAAATGTCGTAATAACCCCGCCCCG | |
| | TTGACGCAAATGGGCGGTAGGCGTGTACGGTGGGAGGT | |
| | TCATATAAGCAGAGCTCGTTTAGTGAACCG | |
| Cytomegalo- | TAGTTATTAATAGTAATCAATTACGGGGTCATTAGTTC | 30 |
| virus (CMV) | ATAGCCCATATATGGAGTTCCGCGTTACATAACTTACG | |
| (second | GTAAATGGCCCGCCTGGCTGACCGCCCAACGACCCCCG | |
| version) | CCCATTGACGTCAATAATGACGTATGTTCCCATAGTAA | |
| | CGCCAATAGGGACTTTCCATTGACGTCAATGGGTGGAG | |
| | TATTTACGGTAAACTGCCCACTTGGCAGTACATCAAGT | |
| | GTATCATATGCCAAGTACGCCCCCTATTGACGTCAATG | |
| | ACGGTAAATGGCCCGCCTGGCATTATGCCCAGTACATG | |
| | ACCTTATGGGACTTTCCTACTTGGCAGTACATCTACGT | |
| | ATTAGTCATCGCTATTACCATGGTGATGCGGTTTTGGC | |
| | AGTACATCAATGGGCGTGGATAGCGGTTTGACTCACGG | |
| | GGATTTCCAAGTCTCCACCCCATTGACGTCAATGGGAG | |
| | TTTGTTTTGGCACCAAAATCAACGGGACTTTCCAAAAT | |
| | GTCGTAACAACTCCGCCCCATTGACGCAAATGGGCGGT | |
| | AGGCGTGTACGGTGGGAGGTCTATATAAGCAGAGCTGG | |
| | TTTAGTGAACCGT | |
| Cytomegalo- | CGTTACATAACTTACGGTAAATGGCCCGCCTGGCTGAC | 31 |
| virus (CMV) | CGCCCAACGACCCCCGCCCATTGACGTCAATAATGACG | |
| (third | TATGTTCCCATAGTAACGCCAATAGGGACTTTCCATTG | |
| version) | ACGTCAATGGGTGGAGTATTTACGGTAAACTGCCCACT | |
| | TGGCAGTACATCAAGTGTATCATATGCCAAGTACGCCC | |
| | CCTATTGACGTCAATGACGGTAAATGGCCCGCCTGGCA | |
| | TTATGCCCAGTACATGACCTTATGGGACTTTCCTACTT | |
| | GGCAGTACATCTACGTATTAGTCATCGCTATTACCATG | |
| | GTGATGCGGTTTTGGCAGTACATCAATGGGCGTGGATA | |
| | GCGGTTTGACTCACGGGGATTTCCAAGTCTCCACCCCA | |
| | TTGACGTCAATGGGAGTTTGTTTTGGCACCAAAATCAA | |
| | CGGGACTTTCCAAAATGTCGTAACAACTCCGCCCCATT | |
| | GACGCAAATGGGCGGTAGGCGTGTACGGTGGGAGGTCT | |
| | ATATAAGCAGAGCT | |
| CAG promoter | ACTTACGGTAAATGGCCCGCCTGGCTGACCGCCCAACG | 32 |
| (first | ACCCCCGCCCATTGACGTCAATAATGACGTATGTTCCC | |
| version) | ATAGTAACGCCAATAGGGACTTTCCATTGACGTCAATG | |
| | GGTGGAGTATTTACGGTAAACTGCCCACTTGGCAGTAC | |
| | ATCAAGTGTATCATATGCCAAGTACGCCCCCTATTGAC | |
| | GTCAATGACGGTAAATGGCCCGCCTGGCATTATGCCCA | |
| | GTACATGACCTTATGGGACTTTCCTACTTGGCAGTACA | |
| | TCTACGTATTAGTCATCGCTATTACCATGGTCGAGGTG | |
| | AGCCCCACGTTCTGCTTCACTCTCCCCATCTCCCCCCC | |
| | CTCCCCACCCCCAATTTTGTATTTATTTATTTTTTAAT | |
| | TATTTTGTGCAGCGATGGGGGCGGGGGGGGGGGGGGCG | |
| | CGCGCCAGGCGGGGCGGGGCGGGGCGAGGGGCGGGGCG | |
| | GGGCGAGGCGGAGAGGTGCGGCGGCAGCCAATCAGAGC | |
| | GGCGCGCTCCGAAAGTTTCCTTTTATGGCGAGGCGGCG | |
| | GCGGCGGCGGCCCTATAAAAAGCGAAGCGCGCGGCGGG | |
| | CGG | |
| CAG promoter | CGTTACATAACTTACGGTAAATGGCCCGCCTGGCTGAC | 33 |
| (second | CGCCCAACGACCCCCGCCCATTGACGTCAATAATGACG | |
| version) | TATGTTCCCATAGTAACGCCAATAGGGACTTTCCATTG | |
| | ACGTCAATGGGTGGAGTATTTACGGTAAACTGCCCACT | |
| | TGGCAGTACATCAAGTGTATCATATGCCAAGTACGCCC | |
| | CCTATTGACGTCAATGACGGTAAATGGCCCGCCTGGCA | |
| | TTATGCCCAGTACATGACCTTATGGGACTTTCCTACTT | |
| | GGCAGTACATCTACGTATTAGTCATCGCTATTACCATG | |
| | TCGAGGTGAGCCCCACGTTCTGCTTCACTCTCCCCATC | |
| | TCCCCCCCCTCCCCACCCCCAATTTTGTATTTATTTAT | |
| | TTTTTAATTATTTTGTGCAGCGATGGGGGCGGGGGGGG | |
| | GGGGGGCGCGCGCCAGGCGGGGCGGGGCGGGGCGAGGG | |
| | GCGGGGCGGGGCGAGGCGGAGAGGTGCGGCGGCAGCCA | |
| | ATCAGAGCGGCGCGCTCCGAAAGTTTCCTTTTATGGCG | |
| | AGGCGGCGGCGGCGGCGGCCCTATAAAAAGCGAAGCGC | |
| | GCGGCGGGCG | |
| Human EF1- | CAACCTTTGGAGCTAAGCCAGCAATGGTAGAGGGAAGA | 34 |
| alpha | TTCTGCACGTCCCTTCCAGGCGGCCTCCCCGTCACCAC | |
| (EF1-α) | CCCCCCCAACCCGCCCCGACCGGAGCTGAGAGTAATTC | |
| | ATACAAAAGGACTCGCCCCTGCCTTGGGGAATCCCAGG | |
| | GACCGTCGTTAAACTCCCACTAACGTAGAACCCAGAGA | |
| | TCGCTGCGTTCCCGCCCCCTCACCCGCCCGCTCTCGTC | |
| | ATCACTGAGGTGGAGAATAGCATGCGTGAGGCTCCGGT | |
| | GCCCGTCAGTGGGCAGAGCGCACATCGCCCACAGTCCC | |
| | CGAGAAGTTGGGGGGAGGGGTCGGCAATTGAACGGGTG | |
| | CCTAGAGAAGGTGGCGCGGGGTAAACTGGGAAAGTGAT | |
| | GTCGTGTACTGGCTCCGCCTTTTTCCCGAGGGTGGGGG | |
| | AGAACCGTATATAAGTGCAGTAGTCGCCGTGAACGTT | |
| Human | ACTTGTGGACAAAGTTTGCTCTATTCCACCTCCTCCAG | 35 |
| CamKIIa | GCCCTCCTTGGGTCCATCACCCCAGGGGTGCTGGGTCC | |
| (CaMKIIa) | ATCCCACCCCCAGGCCCACACAGGCTTGCAGTATTGTG | |
| | TGCGGTATGGTCAGGGCGTCCGAGAGCAGGTTTCGCAG | |
| | TGGAAGGCAGGCAGGTGTTGGGGAGGCAGTTACCGGGG | |
| | CAACGGGAACAGGGCGTTTTGGAGGTGGTTGCCATGGG | |
| | GACCTGGATGCTGACGAAGGCTCGCGAGGCTGTGAGCA | |
| | GCCACAGTGCCCTGC | |

Further illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. A large variety of other promoters are known and generally available in the art, and the sequences of many such promoters are available in sequence databases such as the GenBank database.

### Other Regulatory Elements

In some cases, vectors of the present disclosure further comprise one or more regulatory elements selected from the group consisting of an enhancer, an intron, a poly-A signal, a 2A peptide encoding sequence, a WPRE (Woodchuck hepatitis virus posttranscriptional regulatory element), and a HPRE (Hepatitis B posttranscriptional regulatory element).

In some embodiments, the vector comprises a CMV enhancer.

In certain embodiments, the vectors comprise one or more enhancers. In particular embodiments, the enhancer is a CMV enhancer sequence, a GAPDH enhancer sequence, a β-actin enhancer sequence, or an EF1-α enhancer sequence. Sequences of the foregoing are known in the art. For example, the sequence of the CMV immediate early (IE) enhancer is:

(SEQ ID NO: 36)
ACTTACGGTAAATGGCCCGCCTGGCTGACCGCCCAACGACCCCCGCCCAT
TGACGTCAATAATGACGTATGTTCCCATAGTAACGCCAATAGGGACTTTC
CATTGACGTCAATGGGTGGAGTATTTACGGTAAACTGCCCACTTGGCAGT
ACATCAAGTGTATCATATGCCAAGTACGCCCCCTATTGACGTCAATGACG
GTAAATGGCCCGCCTGGCATTATGCCCAGTACATGACCTTATGGGACTTT
CCTACTTGGCAGTACATCTACGTATTAGTCATCGCTATTACCA.

In certain embodiments, the vector comprises an enhancer that is linked to a promoter. For example, the vector may comprise an MHCK7 promoter and enhancer. In certain embodiments, the MHCK7 promoter and enhancer is at least 75%, 80%, 85%, 90%, 92%, 93%, 94%, 95%, 96%, 97%, 98%, 99%, or 100% identical to the following sequence:

(SEQ ID NO: 21)
ACCCTTCAGATTAAAAATAACTGAGGTAAGGGCCTGGGTAGGGGAGGTGG
TGTGAGACGCTCCTGTCTCTCCTCTATCTGCCCATCGGCCCTTTGGGGAG
GAGGAATGTGCCCAAGGACTAAAAAAAGGCCATGGAGCCAGAGGGGCGAG
GGCAACAGACCTTTCATGGGCAAACCTTGGGGCCCTGCTGTCTAGCATGC
CCCACTACGGGTCTAGGCTGCCCATGTAAGGAGGCAAGGCCTGGGGACAC
CCGAGATGCCTGGTTATAATTAACCCAGACATGTGGCTGCCCCCCCCCCC
CCAACACCTGCTGCCTCTAAAAATAACCCTGTCCCTGGTGGATCCCCTGC
ATGCGAAGATCTTCGAACAAGGCTGTGGGGGACTGAGGGCAGGCTGTAAC
AGGCTTGGGGGCCAGGGCTTATACGTGCCTGGGACTCCCAAAGTATTACT
GTTCCATGTTCCCGGCGAAGGGCCAGCTGTCCCCCGCCAGCTAGACTCAG
CACTTAGTTTAGGAACCAGTGAGCAAGTCAGCCCTTGGGGCAGCCCATAC
AAGGCCATGGGGCTGGGCAAGCTGCACGCCTGGGTCCGGGGTGGGCACGG
TGCCCGGGCAACGAGCTGAAAGCTCATCTGCTCTCAGGGGCCCCTCCCTG
GGGACAGCCCCTCCTGGCTAGTCACACCCTGTAGGCTCCTCTATATAACC
CAGGGGCACAGGGGCTGCCCTCATTCTACCACCACCTCCACAGCACAGAC
AGACACTCAGGAGCCAGCCAG.

In certain embodiments, the vectors comprise one or more introns. In particular embodiments, the intron is a rabbit globin intron sequence, a chicken β-actin intron sequence, a synthetic intron sequence, an SV40 intron, or an EF1-α intron sequence.

In certain embodiments, the vectors comprise a polyA sequence. In particular embodiments, the polyA sequence is a rabbit globin polyA sequence, a human growth hormone polyA sequence, a bovine growth hormone polyA sequence, a PGK polyA sequence, an SV40 polyA sequence, or a TK polyA sequence. In some embodiments, the poly-A signal may be a bovine growth hormone polyadenylation signal (bGHpA).

In certain embodiments, the vectors comprise one or more transcript stabilizing element. In particular embodiments, the transcript stabilizing element is a WPRE sequence, a HPRE sequence, a scaffold-attachment region, a 3′ UTR, or a 5′ UTR. In particular embodiments, the vectors comprise both a 5′ UTR and a 3′ UTR.

In some embodiments, the vector comprises a 5′ untranslated region (UTR) selected from Table 4. In some embodiments, the vector genome comprises a polynucleotide sequence at least 75%, 80%, 85%, 90%, 92%, 93%, 94%, 95%, 96%, 97%, 98%, 99%, or 100% identical to any one of SEQ ID NOS 38-48.

TABLE 4

| 5′ UNTRANSLATED | | SEQ ID |
|---|---|---|
| REGION | SEQUENCE | NO: |
| Human | CGCGTCCGCCCGCGAGCACAGAGCCTCGCCTTTGCCG | 38 |
| beta-actin | ATCCGCCGCCCGTCCACACCCGCCGCCAGGTAAGCCC | |
| exon/intron | GGCCAGCCGACCGGGGCATGCGGCCGCGGCCCTTCGC | |
| | CCGTGCAGAGCCGCCGTCTGGGCCGCAGCGGGGGGCG | |
| | CATGGGGCGGAACCGGACCGCCGTGGGGGGCGCGGGA | |
| | GAAGCCCCTGGGCCTCCGGAGATGGGGGACACCCCAC | |
| | GCCAGTTCGCAGGCGCGAGGCCGCGCTCGGGCGGGCG | |
| | CGCTCCGGGGGTGCCGCTCTCGGGGCGGGGGCAACCG | |
| | GCGGGGTCTTTGTCTGAGCCGGGCTCTTGCCAATGGG | |
| | GATCGCACGGTGGGCGCGGCGTAGCCCCCGTCAGGCC | |
| | CGGTGGGGGCTGGGGCGCCATGCGCGTGCGCGCTGGT | |
| | CCTTTGGGCGCTAACTGCGTGCGCGCTGGGAATTGGC | |
| | GCTAATTGCGCGTGCGCGCTGGGACTCAATGGCGCTA | |
| | ATCGCGCGTGCGTTCTGGGGCCCGGGCGCTTGCGCCA | |
| | CTTCCTGCCCGAGCCGCTGGCGCCCGAGGGTGTGGCC | |
| | GCTGCGTGCGCGCGCGCGACCCGGTCGCTGTTTGAAC | |
| | CGGGCGGAGGCGGGGCTGGCGCCCGGTTGGGAGGGGG | |
| | TTGGGGCCTGGCTTCCTGCCGCGCGCCGCGGGGACGC | |
| | CTCCGACCAGTGTTTGCCTTTTATGGTAATAACGCGG | |
| | CCGGCCCGGCTTCCTTTGTCCCCAATCTGGGCGCGCG | |
| | CCGGCGCCCCCTGGCGGCCTAAGGACTCGGCGCGCCG | |
| | GAAGTGGCCAGGGCGGCAGCGGCTGCTCTTGGCGGCC | |
| | CCGAGGTGACTATAGCCTTCTTTTGTGTCTTGATAGT | |
| | TCGCCAGCCTCTGCTAACCATGTTCATGCCTTCTTCT | |
| | TTTTCCTACAGCTCCTGGGCAACGTGCTGGTTATTGT | |
| | GCTGTCTCATCATTTTGGCAAAGAATTC | |
| Chicken | GTCGCTGCGCGCTGCCTTCGCCCCGTGCCCCGCTCCG | 39 |
| beta-actin | CCGCCGCCTCGCGCCGCCCGCCCCGGCTCTGACTGAC | |
| exon/intron + | CGCGTTACTCCCACAGGTGAGCGGGCGGGACGGCCCT | |
| rabbit globin | TCTCCTCCGGGCTGTAATTAGCGCTTGGTTTAATGAC | |
| intron | GGCTTGTTTCTTTTCTGTGGCTGCGTGAAAGCCTTGA | |
| | GGGGCTCCGGGAGGGCCCTTTGTGCGGGGGGAGCGGC | |
| | TCGGGGGGTGCGTGCGTGTGTGTGTGCGTGGGGAGCG | |
| | CCGCGTGCGGCTCCGCGCTGCCCGGCGGCTGTGAGCG | |
| | CTGCGGGCGCGGCGCGGGGCTTTGTGCGCTCCGCAGT | |
| | GTGCGCGAGGGGAGCGCGGCCGGGGGCGGTGCCCCGC | |
| | GGTGCGGGGGGGGCTGCGAGGGGAACAAAGGCTGCGT | |
| | GCGGGGTGTGTGCGTGGGGGGGTGAGCAGGGGGTGTG | |
| | GGCGCGTCGGTCGGGCTGCAACCCCCCCTGCACCCCC | |
| | CTCCCCGAGTTGCTGAGCACGGCCCGGCTTCGGGTGC | |
| | GGGGCTCCGTACGGGGCGTGGCGCGGGGCTCGCCGTG | |
| | CCGGGCGGGGGGTGGCGGCAGGTGGGGGTGCCGGGCG | |
| | GGGGGGGCCGCCTCGGGCCGGGGAGGGCTCGGGGGAG | |
| | GGGCGCGGCGGCCCCCGGAGCGCCGGCGGCTGTCGAG | |
| | GCGCGGCGAGCCGCAGCCATTGCCTTTTATGGTAATC | |
| | GTGCGAGAGGGCGCAGGGACTTCCTTTGTCCCAAATC | |
| | TGTGCGGAGCCGAAATCTGGGAGGCGCCGCCGCACCC | |
| | CCTCTAGCGGGCGCGGGGCGAAGCGGTGCGGCGCCGG | |
| | CAGGAAGGAAATGGGCGGGGAGGGCCTTCGTGCGTCG | |
| | CCGCGCCGCCGTCCCCTTCTCCCTCTCCAGCCTCGGG | |
| | GCTGTCCGCGGGGGGACGGCTGCCTTCGGGGGGGACG | |
| | GGGCAGGGCGGGGTTCGGCTTCTGGCGTGTGACCGGC | |
| | GGCTCTAGAGCCTCTGCTAACCATGTTCATGCCTTCT | |
| | TCTTTTTCCTACAGCTCCTGGGCAACGTGCTGGTTAT | |
| | TGTGCTGTCTCATCATTTTGGCAAAGAATTC | |
| Chimeric | GGTAAGTTTAGTCTTTTTGTCTTTTATTTCAGGTCCC | 40 |
| intron | GGATCCGGTGGTGGTGCAAATCAAAGAACTGCTCCTC | |
| sequence | AGTGGATGTTGCCTTTACTTCTAGGCCTGTACGGAAG | |
| | TGTTACTTCTGCTCTAAAAGCTGCGGAATTGTACCCG | |
| | C | |
| 5′ UTR-Syn1 | AGTCTGCGGTGGGCAGCGGAGGAGTCGTGTCGTGCCT | 41 |
| Hs | GAGAGCGCAGCTGTGCTCCTGGGCACCGCGCAGTCCG | |
| | CCCCCGCGGCTCCTGGCCAGACCACCCCTAGGACCCC | |
| | CTGCCCCAAGTCGCA | |
| CMV IE exon | TCAGATCGCCTGGAGAGGCCATCCACGCTGTTTTGAC | 42 |
| | CTCCATAGTGGACACCGGGACCGATCCAGCCTCCGCG | |
| | GCCGGGAACGGTGCATTGGAACGCGGATTCCCCGTGC | |
| | CAAGAGTGAC | |
| TPL-ePKP2 | CTCACTCTCTTCCGCATCGCTGTCTGCGAGGGCCAGC | 43 |
| (adenovirus | TGTTGGGCTCGCGGTTGAGGACAAACTCTTCGCGGTC | |
| derived | TTTCCAGTACTCTTGGATCGGAAACCCGTCGGCCTCC | |
| enhancer | GAACGGTACTCCGCCACCGAGGGACCTGAGCGAGTCC | |
| element) | GCATCGACCGGATCGGAAAACCTCTCGAGAAAGGCGT | |
| | CTAACCAGTCACAGTCGCAAGGTAGGCTGAGCACCGT | |
| | GGCGGGCGGCAGCGGGTGGCGGTCGGGGTTGTTTCTG | |
| | GCGGAGGTGCTGCTGATGATGTAATTAAAGTAGGCGG | |
| | TCTTGAGACGGCGGATGGTCGAGGTGAGGTGTGGCAG | |
| | GCTTGAGATCCAGCTGTTGGGGTGAGTACTCCCTCTC | |
| | AAAAGCGGGCATTACTTCTGCGCTAAGATTGTCAGTT | |
| | TCCAAAAACGAGGAGGATTTGATATTCACCTGGCCCG | |
| | ATCTGGCCATACACTTGAGTGACAATGACATCCACTT | |
| | TGCCTTTCTCTCCACAGGTGTCCACTCCCAG | |
| Human EF1-α | CTTTTTCGCAACGGGTTTGCCGCCAGAACACAGGTAA | 44 |
| intron/exon | GTGCCGTGTGTGGTTCCCGCGGGCCTGGCCTCTTTAC | |
| | GGGTTATGGCCCTTGCGTGCCTTGAATTACTTCCACC | |
| | TGGCTCCAGTACGTGATTCTTGATCCCGAGCTGGAGC | |
| | CAGGGGCGGGCCTTGCGCTTTAGGAGCCCCTTCGCCT | |
| | CGTGCTTGAGTTGAGGCCTGGCCTGGGCGCTGGGGCC | |
| | GCCGCGTGCGAATCTGGTGGCACCTTCGCGCCTGTCT | |
| | CGCTGCTTTCGATAAGTCTCTAGCCATTTAAAATTTT | |
| | TGATGACGTGCTGCGACGCTTTTTTTCTGGCAAGATA | |
| | GTCTTGTAAATGCGGGCCAGGATCTGCACACTGGTAT | |
| | TTCGGTTTTTGGGCCCGCGGCCGGCGACGGGGCCCGT | |
| | GCGTCCCAGCGCACATGTTCGGCGAGGCGGGGCCTGC | |
| | GAGCGCGGCCACCGAGAATCGGACGGGGGTAGTCTCA | |
| | AGCTGGCCGGCCTGCTCTGGTGCCTGGCCTCGCGCCG | |
| | CCGTGTATCGCCCCGCCCTGGGCGGCAAGGCTGGCCC | |
| | GGTCGGCACCAGTTGCGTGAGCGGAAAGATGGCCGCT | |
| | TCCCGGCCCTGCTCCAGGGGGCTCAAAATGGAGGACG | |
| | CGGCGCTCGGGAGAGCGGGGGGTGAGTCACCCACACA | |
| | AAGGAAAAGGGCCTTTCCGTCCTCAGCCGTCGCTTCA | |
| | TGTGACTCCACGGAGTACCGGGCGCCGTCCAGGCACC | |
| | TCGATTAGTTCTGGAGCTTTTGGAGTACGTCGTCTTT | |
| | AGGTTGGGGGGAGGGGTTTTATGCGATGGAGTTTCCC | |
| | CACACTGAGTGGGTGGAGACTGAAGTTAGGCCAGCTT | |
| | GGCACTTGATGTAATTCTCCTTGGAATTTGGCCTTTT | |
| | TGAGTTTGGATCTTGGTTCATTCTCAAGCCTCAGACA | |
| | GTGGTTCAAAGTTTTTTTCTTCCATTTCAG | |
| Human | GTAAGTGCCGTGTGTGGTTCCCGCGGGCCTGGCCTCT | 45 |
| EF1-α, | TTACGGGTTATGGCCCTTGCGTGCCTTGAATTACTTC | |
| intron A | CACCTGGCTGCAGTACGTGATTCTTGATCCCGAGCTT | |
| | CGGGTTGGAAGTGGGTGGGAGAGTTCGAGGCCTTGCG | |
| | CTTAAGGAGCCCCTTCGCCTCGTGCTTGAGTTGAGGC | |
| | CTGGCCTGGGCGCTGGGGCCGCCGCGTGCGAATCTGG | |
| | TGGCACCTTCGCGCCTGTCTCGCTGCTTTCGATAAGT | |
| | CTCTAGCCATTTAAAATTTTTGATGACCTGCTGCGAC | |
| | GCTTTTTTTCTGGCAAGATAGTCTTGTAAATGCGGGC | |
| | CAAGATCTGCACACTGGTATTTCGGTTTTTGGGGCCG | |
| | CGGGCGGCGACGGGGCCCGTGCGTCCCAGCGCACATG | |
| | TTCGGCGAGGCGGGGCCTGCGAGCGCGGCCACCGAGA | |
| | ATCGGACGGGGGTAGTCTCAAGCTGGCCGGCCTGCTC | |
| | TGGTGCCTGGCCTCGCGCCGCCGTGTATCGCCCCGCC | |
| | CTGGGCGGCAAGGCTGGCCCGGTCGGCACCAGTTGCG | |
| | TGAGCGGAAAGATGGCCGCTTCCCGGCCCTGCTGCAG | |
| | GGAGCTCAAAATGGAGGACGCGGCGCTCGGGAGAGCG | |
| | GGCGGGTGAGTCACCCACACAAAGGAAAAGGGCCTTT | |
| | CCGTCCTCAGCCGTCGCTTCATGTGACTCCACGGAGT | |
| | ACCGGGCGCCGTCCAGGCACCTCGATTAGTTCTCGAG | |
| | CTTTTGGAGTACGTCGTCTTTAGGTTGGGGGGAGGGG | |
| | TTTTATGCGATGGAGTTTCCCCACACTGAGTGGGTGG | |
| | AGACTGAAGTTAGGCCAGCTTGGCACTTGATGTAATT | |
| | CTCCTTGGAATTTGCCCTTTTTGAGTTTGGATCTTGG | |
| | TTCATTCTCAAGCCTCAGACAGTGGTTCAAAGTTTTT | |
| | TTCTTCCATTTCAG | |
| 5′ UTR | TCAGAAGCCCCGGGCTCGTCAGTCAAACCGGTTCTCT | 46 |
| human | GTTTGCACTCGGCAGCACGGGCAGGCAAGTGGTCCCT | |
| CamKIIa | AGGTTCGGG | |
| B-globin | GTGAGTCTATGGGACCCTTGATGTTTTCTTTCCCCTT | 47 |
| intron | CTTTTCTATGGTTAAGTTCATGTCATAGGAAGGGGAG | |
| | AAGTAACAGGGTACACATATTGACCAAATCAGGGTAA | |
| | TTTTGCATTTGTAATTTTAAAAAATGCTTTCTTCTTT | |
| | TAATATACTTTTTTGTTTATCTTATTTCTAATACTTT | |
| | CCCTAATCTCTTTCTTTCAGGGCAATAATGATACAAT | |
| | GTATCATGCCTCTTTGCACCATTCTAAAGAATAACAG | |
| | TGATAATTTCTGGGTTAAGGCAATAGCAATATTTCTG | |
| | CATATAAATATTTCTGCATATAAATTGTAACTGATGT | |
| | AAGAGGTTTCATATTGCTAATAGCAGCTACAATCCAG | |
| | CTACCATTCTGCTTTTATTTTATGGTTGGGATAAGGC | |
| | TGGATTATTCTGAGTCCAAGCTAGGCCCTTTTGCTAA | |
| | TCATGTTCATACCTCTTATCTTCCTCCCACAG | |
| SV40 | TCTAGAGGATCCGGTACTCGAGGAACTGAAAAACCAG | 48 |
| intron | AAAGTTAACTGGTAAGTTTAGTCTTTTTGTCTTTTAT | |
| | TTCAGGTCCCGGATCCGGTGGTGGTGCAAATCAAAGA | |
| | ACTGCTCCTCAGTGGATGTTGCCTTTACTTCTAGGCC | |
| | TGTACGGAAGTGTTACTTCTGCTCTAAAAGCTGCGGA | |
| | ATTGTACCCGC | |

In some embodiments, the vector comprises a 3′ untranslated region selected from Table 5. In some embodiments, the vector genome comprises a polynucleotide sequence at least 75%, 80%, 85%, 90%, 92%, 93%, 94%, 95%, 96%, 97%, 98%, 99%, or 100% identical to any one of SEQ ID NOS 49-57.

TABLE 5

| 3′ UNTRANSLATED | | SEQ ID |
|---|---|---|
| REGION | SEQUENCE | NO: |
| WPRE(x) (mutated | AATCAACCTCTGGATTACAAAATTTGTGAAAGATTG | 49 |
| woodchuck hepatitis | ACTGGTATTCTTAACTATGTTGCTCCTTTTACGCTA | |
| regulatory element - | TGTGGATACGCTGCTTTAATGCCTTTGTATCATGCT | |
| version 1) | ATTGCTTCCCGTATGGCTTTCATTTTCTCCTCCTTG | |
| | TATAAATCCTGGTTGCTGTCTCTTTATGAGGAGTTG | |
| | TGGCCCGTTGTCAGGCAACGTGGCGTGGTGTGCACT | |
| | GTGTTTGCTGACGCAACCCCCACTGGTTGGGGCATT | |
| | GCCACCACCTGTCAGCTCCTTTCCGGGACTTTCGCT | |
| | TTCCCCCTCCCTATTGCCACGGCGGAACTCATCGCC | |
| | GCCTGCCTTGCCCGCTGCTGGACAGGGGCTCGGCTG | |
| | TTGGGCACTGACAATTCCGTGGTGTTGTCGGGGAAA | |
| | TCATCGTCCTTTCCTTGGCTGCTCGCCTGTGTTGCC | |
| | ACCTGGATTCTGCGCGGGACGTCCTTCTGCTACGTC | |
| | CCTTCGGCCCTCAATCCAGCGGACCTTCCTTCCCGC | |
| | GGCCTGCTGCCGGCTCTGCGGCCTCTTCCGCGTCTT | |
| | CGCCTTCGCCCTCAGACGAGTCGGATCTCCCTTTGG | |
| | GCCGCCTCCCCGC | |
| WPRE(x) (mutated | TCAACCTCTGGATTACAAAATTTGTGAAAGATTGAC | 50 |
| woodchuck hepatitis | TGGTATTCTTAACTATGTTGCTCCTTTTACGCTATG | |
| regulatory element - | TGGATACGCTGCTTTAATGCCTTTGTATCATGCTAT | |
| version 2) | TGCTTCCCGTATGGCTTTCATTTTCTCCTCCTTGTA | |
| | TAAATCCTGGTTGCTGTCTCTTTATGAGGAGTTGTG | |
| | GCCCGTTGTCAGGCAACGTGGCGTGGTGTGCACTGT | |
| | GTTTGCTGACGCAACCCCCACTGGTTGGGGCATTGC | |
| | CACCACCTGTCAGCTCCTTTCCGGGACTTTCGCTTT | |
| | CCCCCTCCCTATTGCCACGGCGGAACTCATCGCCGC | |
| | CTGCCTTGCCCGCTGCTGGACAGGGGCTCGGCTGTT | |
| | GGGCACTGACAATTCCGTGGTGTTGTCGGGGAAATC | |
| | ATCGTCCTTTCCTTGGCTGCTCGCCTGTGTTGCCAC | |
| | CTGGATTCTGCGCGGGACGTCCTTCTGCTACGTCCC | |
| | TTCGGCCCTCAATCCAGCGGACCTTCCTTCCCGCGG | |
| | CCTGCTGCCGGCTCTGCGGCCTCTTCCGCGTCTTCG | |
| | CCTTCGCCCTCAGACGAGTCGGATCTCCCTTTGGGC | |
| | CGCCTCCCCGCA | |
| WPRE(x) (mutated | TTCCTGTTAATCAACCTCTGGATTACAAAATTTGTG | 51 |
| woodchuck hepatitis | AAAGATTGACTGGTATTCTTAACTATGTTGCTCCTT | |
| regulatory element - | TTACGCTATGTGGATACGCTGCTTTAATGCCTTTGT | |
| version 3) | ATCATGCTATTGCTTCCCGTATGGCTTTCATTTTCT | |
| | CCTCCTTGTATAAATCCTGGTTGCTGTCTCTTTATG | |
| | AGGAGTTGTGGCCCGTTGTCAGGCAACGTGGCGTGG | |
| | TGTGCACTGTGTTTGCTGACGCAACCCCCACTGGTT | |
| | GGGGCATTGCCACCACCTGTCAGCTCCTTTCCGGGA | |
| | CTTTCGCTTTCCCCCTCCCTATTGCCACGGCGGAAC | |
| | TCATCGCCGCCTGCCTTGCCCGCTGCTGGACAGGGG | |
| | CTCGGCTGTTGGGCACTGACAATTCCGTGGTGTTGT | |
| | CGGGGAAGCTGACGTCCTTTCCGCGGCTGCTCGCCT | |
| | GTGTTGCCACCTGGATTCTGCGCGGGACGTCCTTCT | |
| | GCTACGTCCCTTCGGCCCTCAATCCAGCGGACCTTC | |
| | CTTCCCGCGGCCTGCTGCCGGCTCTGCGGCCTCTTC | |
| | CGCCTCTTCGCCTTCGCCCTCAGACGAGTCGGATCT | |
| | CCCTTTGGGCCGCCTCCCCGCCCATGTATCTTTTTC | |
| | ACCTGTGCCTTGTTTTTGCCTGTGTTCCGCGTCCTA | |
| | CTTTTCAAGCCTCCAAGCTGTGCCTTGGGCGGCTTT | |
| | GGGGCATGGACATAGATCCCTATAAAGAATTTGGTT | |
| | CATCTTATCAGTTGTTGAATTTTCTTCCTTTGGAC | |
| CAAX | TGTGTGATAATG | 52 |
| EES | CTGTTCTCATCACATCATATCAAGGTTATATACCAT | 53 |
| | CAATATTGCCACAGATGTTACTTAGCCTTTTAATAT | |
| | TTCTCTAATTTAGTGTATATGCAATGATAGTTCTCT | |
| | GATTTCTGAGATTGAGTTTCTCATGTGTAATGATTA | |
| | TTTAGAGTTTCTCTTTCATCTGTTCAAATTTTTGTC | |
| | TAGTTTTATTTTTTACTGATTTGTAAGACTTCTTTT | |
| | TATAATCTGCATATTACAATTCTCTTTACTGGGGTG | |
| | TTGCAAATATTTTCTGTCATTCTATGGCCTGACTTT | |
| | TCTTAATGGTTTTTTAATTTTAAAAATAAGTCTTAA | |
| | TATTCATGCAATCTAATTAACAATCTTTTCTTTGTG | |
| | GTTAGGACTTTGAGTCATAAGAAATTTTTCTCTACA | |
| | CTGAAGTCATGATGGCATGCTTCTATATTATTTTCT | |
| | AAAAGATTTAAAGTTTTGCCTTCTCCATTTAGACTT | |
| | ATAATTCACTGGAATTTTTTTGTGTGTATGGTATGA | |
| | CATATGGGTTCCCTTTTATTTTTTACATATAAATAT | |
| | ATTTCCCTGTTTTTCTAAAAAAGAAAAAGATCATCA | |
| | TTTTCCCATTGTAAAATGCCATATTTTTTTCATAGG | |
| | TCACTTACATATATCAATGGGTCTGTTTCTGAGCTC | |
| | TACTCTATTTTATCAGCCTCACTGTCTATCCCCACA | |
| | CATCTCATGCTTTGCTCTAAATCTTGATATTTAGTG | |
| | GAACATTCTTTCCCATTTTGTTCTACAAGAATATTT | |
| | TTGTTATTGTCTTTGGGCTTTCTATATACATTTTGA | |
| | AATGAGGTTGACAAGTTA | |
| HPRE | ATAACAGGCCTATTGATTGGAAAGTTTGTCAACGAA | 54 |
| | TTGTGGGTCTTTTGGGGTTTGCTGCCCCTTTTACGC | |
| | AATGTGGATATCCTGCTTTAATGCCTTTATATGCAT | |
| | GTATACAAGCAAAACAGGCTTTTACTTTCTCGCCAA | |
| | CTTACAAGGCCTTTCTCAGTAAACAGTATATGACCC | |
| | TTTACCCCGTTGCTCGGCAACGGCCTGGTCTGTGCC | |
| | AAGTGTTTGCTGACGCAACCCCCACTGGTTGGGGCT | |
| | TGGCCATAGGCCATCAGCGCATGCGTGGAACCTTTG | |
| | TGTCTCCTCTGCCGATCCATACTGCGGAACTCCTAG | |
| | CCGCTTGTTTTGCTCGCAGCAGGTCTGGAGCAAACC | |
| | TCATCGGGACCGACAATTCTGTCGTACTCTCCCGCA | |
| | AGTATACATCGTTTCCATGGCTGCTAGGCTGTGCTG | |
| | CCAACTGGATCCTGCGCGGGACGTCCTTTGTTTACG | |
| | TCCCGTCGGCGCTGAATCCCGCGGACGACCCCTCCC | |
| | GGGGCCGCTTGGGGCTCTACCGCCCGCTTCTCCGTC | |
| | TGCCGTACCGTCCGACCACGGGGCGCACCTCTCTTT | |
| | ACGCGGACTCCCCGTCTGTGCCTTCTCATCTGCCGG | |
| | ACCGTGTGCACTTCGCTTCACCTCTGCACGTCGCAT | |
| | GGAGGCCACCGTGAACGCCCACCGGAACCTGCCCAA | |
| | GGTCTTGCATAAGAGGACTCTTGGACTTTCAGCAAT | |
| | GTCATC | |
| R2V17 (HepB | TTCCTGTAAACAGGCCTATTGATTGGAAAGTTTGTC | 55 |
| derived enhancer | AACGAATTGTGGGTCTTTTGGGGTTTGCTGCCCCTT | |
| element) | TTACGCAATGTGGATATCCTGCTTTAATGCCTTTAT | |
| | ATGCATGTATACAAGCAAAACAGGCTTTTACTTTCT | |
| | CGCCAACTTACAAGGCCTTTCTCAGTAAACAGTATA | |
| | TGACCCTTTACCCCGTTGCTCGGCAACGGCCTGGTC | |
| | TGTGCCAAGTGTTTGCTGACGCAACCCCCACTGGTT | |
| | GGGGCTTGGCCATAGGCCATCAGCGCATGCGTGGAA | |
| | CCTTTGTGTCTCCTCTGCCGATCCATACTGCGGAAC | |
| | TCCTAGCCGCTTGTTTTGCTCGCAGCTGGACTGGAG | |
| | CAAACCTCATCGGGACCGACAATTCTGTCGTACTCT | |
| | CCCGCAAGCACTCACCGTTTCCGCGGCTGCTCGCCT | |
| | GTGTTGCCACCTGGATTCTGCGCGGGACGTCCTTCT | |
| | GCTACGTCCCTTCGGCCCTCAATCCAGCGGACCTTC | |
| | CTTCCCGCGGCCTGCTGCCGGCTCTGCGGCCTCTTC | |
| | CGCCTCTTCGCCTTCGCCCTCAGACGAGTCGGATCT | |
| | CCCTTTGGGCCGCCTCCCCGCCCATGTATCTTTTTC | |
| | ACCTGTGCCTTGTTTTTGCCTGTGTTCCGCGTCCTA | |
| | CTTTTCAAGCCTCCAAGCTGTGCCTTGGGCGGCTTT | |
| | GGGGCATGGACATAGATCCCTATAAAGAATTTGGTT | |
| | CATCTTATCAGTTGTTGAATTTTCTTCCTTTGGAC | |
| 3′UTR(globin) | GCTGGAGCCTCGGTAGCCGTTCCTCCTGCCCGCTGG | 56 |
| | GCCTCCCAACGGGCCCTCCTCCCCTCCTTGCACCGG | |
| | CCCTTCCTGGTCTTTGAATAAA | |
| WPRE(r) | ATTCGAGCATCTTACCGCCATTTATTCCCATATTTG | 81 |
| | TTCTGTTTTTCTTGATTTGGGTATACATTTAAATGT | |
| | TAATAAAACAAAATGGTGGGGCAATCATTTACATTT | |
| | TTAGGGATATGTAATTACTAGTTCAGGTGTATTGCC | |
| | ACAAGACAAACATGTTAAGAAACTTTCCCGTTATTT | |
| | ACGCTCTGTTCCTGTTAATCAACCTCTGGATTACAA | |
| | AATTTGTGAAAGATTGACTGATATTCTTAACTATGT | |
| | TGCTCCTTTTACGCTGTGTGGATATGCTGCTTTAAT | |
| | GCCTCTGTATCATGCTATTGCTTCCCGTACGGCTTT | |
| | CGTTTTCTCCTCCTTGTATAAATCCTGGTTGCTGTC | |
| | TCTTTATGAGGAGTTGTGGCCCGTTGTCCGTCAACG | |
| | TGGCGTGGTGTGCTCTGTGTTTGCTGACGCAACCCC | |
| | CACTGGCTGGGGCATTGCCACCACCTGTCAACTCCT | |
| | TTCTGGGACTTTCGCTTTCCCCCTCCCGATCGCCAC | |
| | GGCAGAACTCATCGCCGCCTGCCTTGCCCGCTGCTG | |
| | GACAGGGGCTAGGTTGCTGGGCACTGATAATTCCGT | |
| | GGTGTTGTCGGGGAAGGGCC | |

In some embodiments, the vector comprises a polyadenylation (polyA) signal selected from Table 6. In some embodiments, the polyA signal comprises a polynucleotide sequence at least 75%, 80%, 85%, 90%, 92%, 93%, 94%, 95%, 96%, 97%, 98%, 99%, or 100% identical to any one of SEQ ID NOS 52-56.

TABLE 6

| POLYADENYLATION | | SEQ ID |
|---|---|---|
| SITE | SEQUENCE | NO: |
| Rabbit globin | TGGCTAATAAAGGAAATTTATTTTCATTGCAATAGT | 82 |
| (pAGlobin-Oc) | GTGTTGGAATTTTTTGTGTCTCTCACTCGGAAGAAC | |
| | ATATGGGAGGGCAAATCATTTAAAACATCAGAATGA | |
| | GTATTTGGTTTAGAGTTTGGCAACATATGCCCATAT | |
| | GCTGGCTGCCATGAACAAAGGTTGGCTATAAAGAGG | |
| | TCATCAGTATATGAAACAGCCCCCTGCTGTCCATTC | |
| | CTTATTCCATAGAAAAGCCTTGACTTGAGGTTAGAT | |
| | TTTTTTTATATTTTGTTTTGTGTTATTTTTTTCTTT | |
| | AACATCCCTAAAATTTTCCTTACATGTTTTACTAGC | |
| | CAGATTTTTCCTCCTCTCCTGACTACTCCCAGTCAT | |
| | AGCTGTCCCTCTTCTCTTATGGAGATC | |
| Bovine growth | TTGCCAGCCATCTGTTGTTTGCCCCTCCCCCGTGCC | 83 |
| hormone (pAGH- | TTCCTTGACCCTGGAAGGTGCCACTCCCACTGTCCT | |
| Bt - version 1) | TTCCTAATAAAATGAGGAAATTGCATCGCATTGTCT | |
| | GAGTAGGTGTCATTCTATTCTGGGGGGTGGGGTGGG | |
| | GCAGGACAGCAAGGGGGAGGATTGGGAATACAATAG | |
| | CAGGCATGCTGGGGATGCGGTGGGCTCTATGGGTAC | |
| | CCAGGTGCTGAAGAATTGACCCGGTTCCTCCTGGG | |
| Bovine growth | TTGCCAGCCATCTGTTGTTTGCCCCTCCCCCGTGCC | 84 |
| hormone (pAGH- | TTCCTTGACCCTGGAAGGTGCCACTCCCACTGTCCT | |
| Bt - version 2) | TTCCTAATAAAATGAGGAAATTGCATCGCATTGTCT | |
| | GAGTAGGTGTCATTCTATTCTGGGGGGGGGGTGGGG | |
| | CAGGACAGCAAGGGGGAGGATTGGGAAGACAATAGC | |
| | AGGCATGCTGGGGATGCGGTGGGCTCTATGGGTACC | |
| | CAGGTGCTGAAGAATTGACCCGGTTCCTCCTGGG | |
| Bovine growth | CTGTGCCTTCTAGTTGCCAGCCATCTGTTGTTTGCC | 85 |
| hormone (pAGH- | CCTCCCCCGTGCCTTCCTTGACCCTGGAAGGTGCCA | |
| Bt - version 3) | CTCCCACTGTCCTTTCCTAATAAAATGAGGAAATTG | |
| | CATCGCATTGTCTGAGTAGGTGTCATTCTATTCTGG | |
| | GGGGTGGGGGGGGCAGGACAGCAAGGGGGAGGATTG | |
| | GGAAGACAATAGCAGGCATGCTGGGGATGCGGTGGG | |
| | CTCTATGG | |
| Human growth | CTGCCCGGGTGGCATCCCTGTGACCCCTCCCCAGTG | 86 |
| hormone (pAGH- | CCTCTCCTGGCCCTGGAAGTTGCCACTCCAGTGCCC | |
| Hs) | ACCAGCCTTGTCCTAATAAAATTAAGTTGCATCATT | |
| | TTGTCTGACTAGGTGTCCTTCTATAATATTATGGGG | |
| | TGGAGGGGGGTGGTATGGAGCAAGGGGCCCAAGTTG | |
| | GGAAGAAACCTGTAGGGCCTGC | |

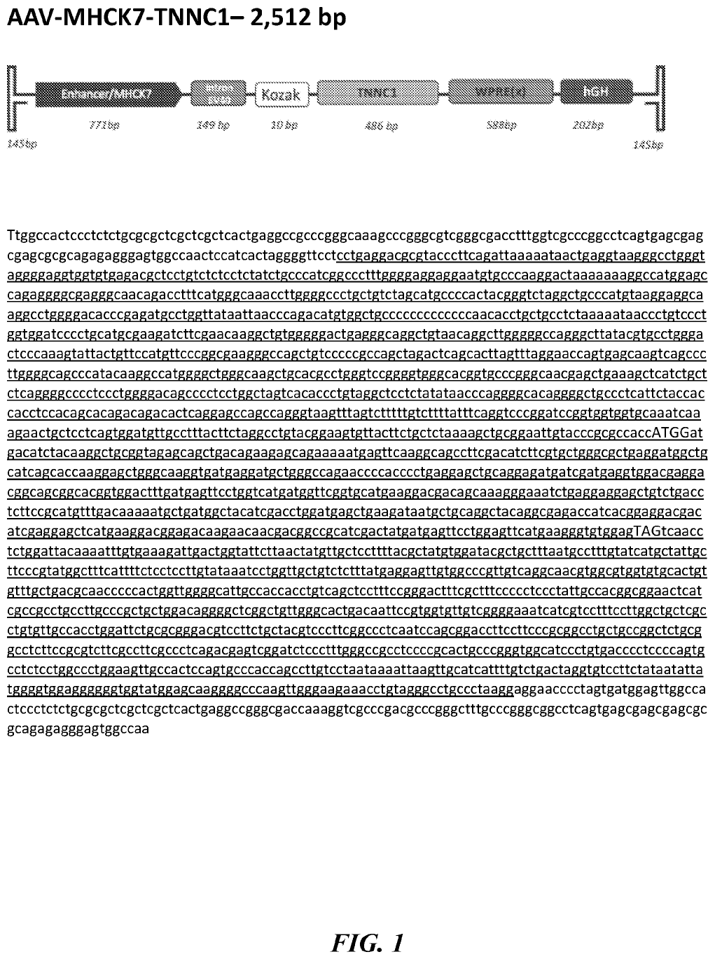

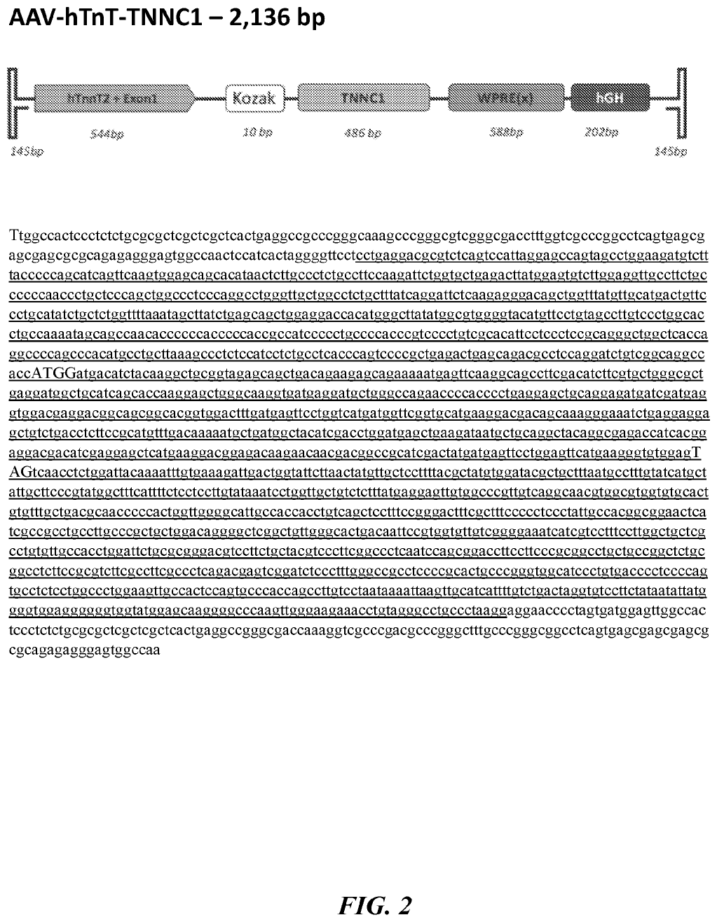

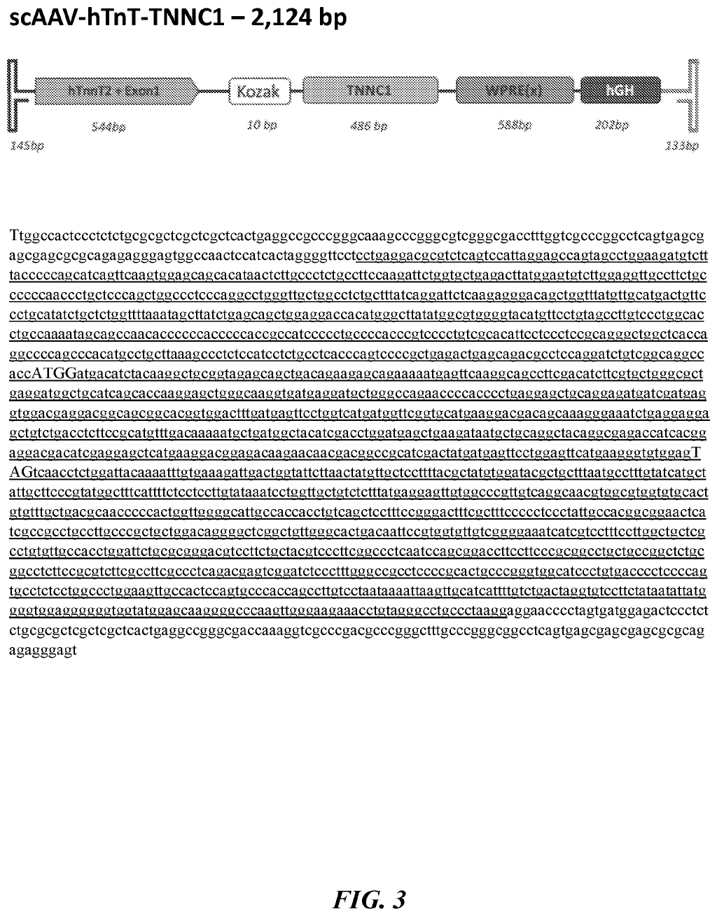

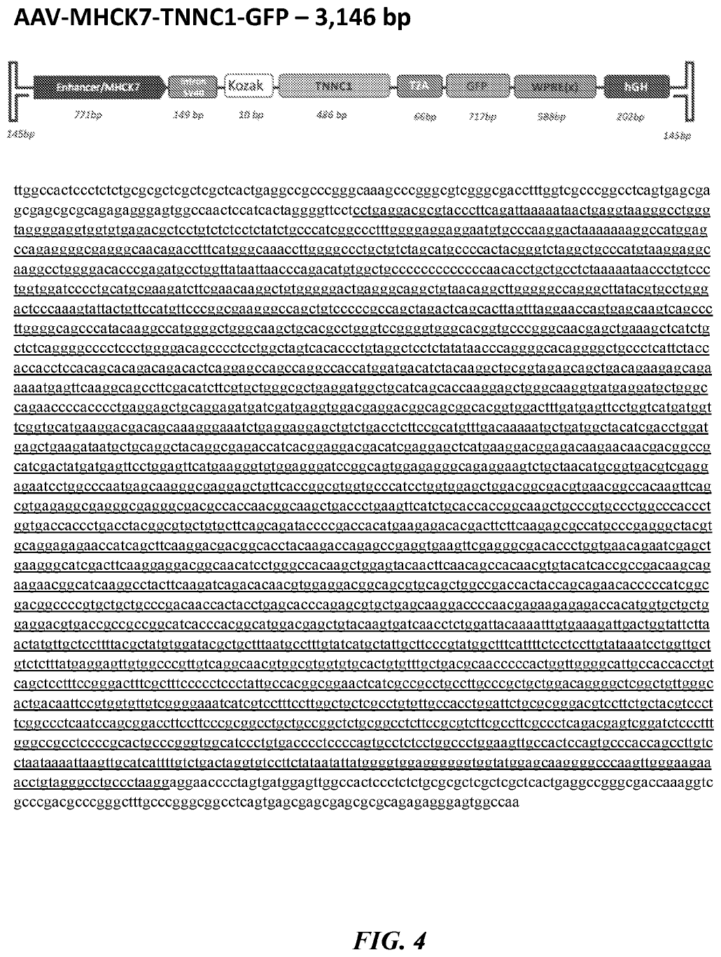

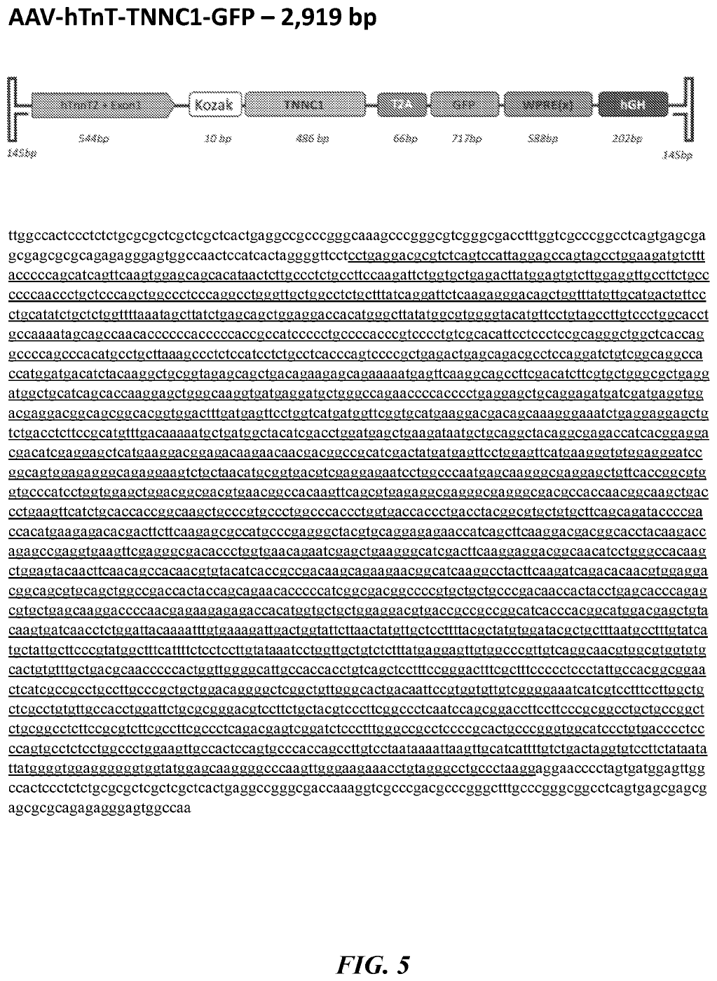

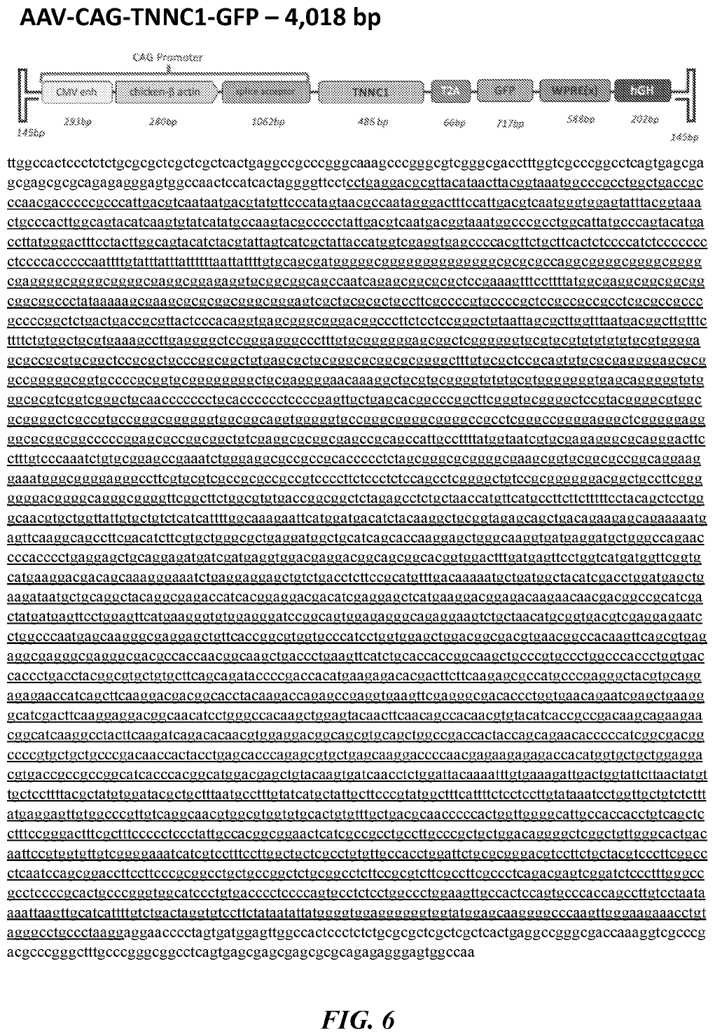

Illustrative vector genomes are depicted in FIGS. 1-6 and provided as SEQ ID NOs: 57-62. The expression cassette of each sequence, shown underlined in FIGS. 1-6, are SEQ ID NOs: 63-68. In some embodiments, the vector genome comprises, consists essentially of, or consists of a polynucleotide sequence that shares at least 90%, 91%, 92%, 93%, 94%, 95%, 96%, 97%, 98%, 99%, or 100% identity to any one of SEQ ID NOs: 57-62, optionally with or without the ITR sequences. In some embodiments, the vector genome comprises, consists essentially of, or consists of a polynucleotide sequence that shares at least 90%, 91%, 92%, 93%, 94%, 95%, 96%, 97%, 98%, 99%, or 100% identity to any one of SEQ ID NOs: 57-62. The disclosure also contemplates expression cassettes of the illustrative vector genomes depicted in FIGS. 1-6 and sequences comprising these, e.g., the sequences set forth in SEQ ID NOs: 57-62, but lacking the 5′ and 3′ ITRs, and variants thereof sharing 90%, 91%, 92%, 93%, 94%, 95%, 96%, 97%, 98%, 99%, or 100% identity to any of the foregoing.

In a preferred embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 57. In a preferred embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 58. In a preferred embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 59. In some embodiments, the vector genome comprises a polynucleotide sequence at least 75%, 80%, 85%, 90%, 92%, 93%, 94%, 95%, 96%, 97%, 98%, 99%, or 100% identical to SEQ ID NO: 60. In a preferred embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 61. In some embodiments, the vector genome comprises a polynucleotide sequence at least 75%, 80%, 85%, 90%, 92%, 93%, 94%, 95%, 96%, 97%, 98%, 99%, or 100% identical to SEQ ID NO: 62.

In a preferred embodiment, the expression cassette comprises a polynucleotide sequence at least 75%, 80%, 85%, 90%, 92%, 93%, 94%, 95%, 96%, 97%, 98%, 99%, or 100% identical to SEQ ID NO: 63. In a preferred embodiment, the expression cassette comprises a polynucleotide sequence at least 75%, 80%, 85%, 90%, 92%, 93%, 94%, 95%, 96%, 97%, 98%, 99%, or 100% identical to SEQ ID NO: 64. In a preferred embodiment, the expression cassette comprises a polynucleotide sequence at least 75%, 80%, 85%, 90%, 92%, 93%, 94%, 95%, 96%, 97%, 98%, 99%, or 100% identical to SEQ ID NO: 65. In some embodiments, the expression cassette comprises a polynucleotide sequence at least 75%, 80%, 85%, 90%, 92%, 93%, 94%, 95%, 96%, 97%, 98%, 99%, or 100% identical to SEQ ID NO: 66. In a preferred embodiment, the expression cassette comprises a polynucleotide sequence at least 75%, 80%, 85%, 90%, 92%, 93%, 94%, 95%, 96%, 97%, 98%, 99%, or 100% identical to SEQ ID NO: 67. In some embodiments, the expression cassette comprises a polynucleotide sequence at least 75%, 80%, 85%, 90%, 92%, 93%, 94%, 95%, 96%, 97%, 98%, 99%, or 100% identical to SEQ ID NO: 68.

### Adeno-Associated Virus Vector and Uses Thereof

Adeno-associated virus (AAV) is a replication-deficient parvovirus, the single-stranded DNA genome of which is about 4.7 kb in length including two ˜145-nucleotide inverted terminal repeat (ITRs). There are multiple known variants of AAV, also sometimes called serotypes when classified by antigenic epitopes. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAVrh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep78, rep68, rep52, and rep40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g., by transfecting packaging cells or by transient transfection with helper plasmids or viruses. Typical examples of virus packaging cells include but are not limited to HeLa cells, SF9 cells (optionally with a baculovirus helper vector), 293 cells, etc. A Herpesvirus-based system can be used to produce AAV vectors, as described in US20170218395A1. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478, the complete contents of each of which is hereby incorporated by reference.

AAV vectors useful in the practice of the present invention can be packaged into AAV virions (viral particles) using various systems including adenovirus-based and helper-free systems. Standard methods in AAV biology include those described in Kwon and Schaffer. Pharm Res. (2008) 25(3):489-99; Wu et al. Mol. Ther. (2006) 14(3):316-27. Burger et al. Mol. Ther. (2004) 10(2):302-17; Grimm et al. Curr Gene Ther. (2003) 3(4):281-304; Deyle D R, Russell D W. Curr Opin Mol Ther. (2009) 11(4):442-447; McCarty et al. Gene Ther. (2001) 8(16):1248-54; and Duan et al. Mol Ther. (2001) 4(4):383-91. Helper-free systems included those described in U.S. Pat. Nos. 6,004,797; 7,588,772; and 7,094,604;

AAV DNA in the rAAV genomes may be from any AAV variant or serotype for which a recombinant virus can be derived including, but not limited to, AAV variants or serotypes AAV-1, AAV-2, AAV-3, AAV-4, AAV-5, AAV-6, AAV-7, AAV-8, AAV-9, AAV-10, AAV-11, AAV-12, AAV-13 and AAVrh10. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

In some cases, the rAAV comprises a self-complementary genome. As defined herein, an rAAV comprising a “self-complementary” or “double stranded” genome refers to an rAAV which has been engineered such that the coding region of the rAAV is configured to form an intra-molecular double-stranded DNA template, as described in McCarty et al. Self-complementary recombinant adeno-associated virus (scAAV) vectors promote efficient transduction independently of DNA synthesis. Gene Therapy. 8 (16): 1248-54 (2001). The present disclosure contemplates the use, in some cases, of an rAAV comprising a self-complementary genome because upon infection (such transduction), rather than waiting for cell mediated synthesis of the second strand of the rAAV genome, the two complementary halves of scAAV will associate to form one double stranded DNA (dsDNA) unit that is ready for immediate replication and transcription. It will be understood that instead of the full coding capacity found in rAAV (4.7-6kb), rAAV comprising a self-complementary genome can only hold about half of that amount (≈2.4kb).

In other cases, the rAAV vector comprises a single stranded genome. As defined herein, a “single standard” genome refers to a genome that is not self-complementary. In most cases, non-recombinant AAVs have singled stranded DNA genomes. There have been some indications that rAAVs should be scAAVs to achieve efficient transduction of cells. The present disclosure contemplates, however, rAAV vectors that maybe have singled stranded genomes, rather than self-complementary genomes, with the understanding that other genetic modifications of the rAAV vector may be beneficial to obtain optimal gene transcription in target cells. In some cases, the present disclosure relates to single-stranded rAAV vectors capable of achieving efficient gene transfer to anterior segment in the mouse eye. See Wang et al. Single stranded adeno-associated virus achieves efficient gene transfer to anterior segment in the mouse eye. PLoS ONE 12(8): e0182473 (2017).

In some cases, the rAAV vector is of the serotype AAV1, AAV2, AAV4, AAV5, AAV6, AAV7, AAV8, AAV9, AAV10, AAV11, AAV12, AAV13, AAVrh10, or AAVrh.74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). In some cases, the rAAV vector is of the serotype AAV9. In some embodiments, said rAAV vector is of serotype AAV9 and comprises a single stranded genome. In some embodiments, said rAAV vector is of serotype AAV9 and comprises a self-complementary genome. In some embodiments, a rAAV vector comprises the inverted terminal repeat (ITR) sequences of AAV2. In some embodiments, the rAAV vector comprises an AAV2 genome, such that the rAAV vector is an AAV-2/9 vector, an AAV-2/6 vector, or an AAV-2/8 vector.

Full-length sequences and sequences for capsid genes for most known AAVs are provided in U.S. Pat. No. 8,524,446, which is incorporated herein in its entirety.

AAV vectors may comprise wild-type AAV sequence or they may comprise one or more modifications to a wild-type AAV sequence. In certain embodiments, an AAV vector comprises one or more amino acid modifications, e.g., substitutions, deletions, or insertions, within a capsid protein, e.g., VP1, VP2 and/or VP3. In particular embodiments, the modification provides for reduced immunogenicity when the AAV vector is provided to a subject.

Capsid proteins of a rAAV may be modified so that the rAAV is targeted to a particular target tissue of interest such as endothelial cells or more particularly endothelial tip cells. In some embodiments, the rAAV is directly injected into the intracerebroventricular space of the subject.

In some embodiments, the rAAV virion is an AAV2 rAAV virion. The capsid many be an AAV2 capsid or functional variant thereof. In some embodiments, the AAV2 capsid shares at least 98%, 99%, or 100% identity to a reference AAV2 capsid, e.g.,

(SEQ ID NO: 69)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL.

In some embodiments, the rAAV virion is an AAV9 rAAV virion. The capsid many be an AAV9 capsid or functional variant thereof. In some embodiments, the AAV9 capsid shares at least 98%, 99%, or 100% identity to a reference AAV9 capsid, e.g.,

(SEQ ID NO: 70)
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL.

In some embodiments, the rAAV virion is an AAV6 rAAV virion. The capsid many be an AAV9 capsid or functional variant thereof. In some embodiments, the AAV6 capsid shares at least 98%, 99%, or 100% identity to a reference AAV6 capsid, e.g.,

(SEQ ID NO: 71)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPFGLVEEGAKTAPGKKRPVEQSP
QEPDSSSGIGKTGQQPAKKRLNFGQTGDSESVPDPQPLGEPPATPAAVGP
TTMASGGGAPMADNNEGADGVGNASGNWHCDSTWLGDRVITTSTRTWALP
TYNNHLYKQISSASTGASNDNHYFGYSTPWGYFDFNRFHCHFSPRDWQRL
INNNWGFRPKRLNFKLFNIQVKEVTTNDGVTTIANNLTSTVQVFSDSEYQ
LPYVLGSAHQGCLPPFPADVFMIPQYGYLTLNNGSQAVGRSSFYCLEYFP
SQMLRTGNNFTFSYTFEDVPFHSSYAHSQSLDRLMNPLIDQYLYYLNRTQ
NQSGSAQNKDLLFSRGSPAGMSVQPKNWLPGPCYRQQRVSKTKTDNNNSN
FTWTGASKYNLNGRESIINPGTAMASHKDDKDKFFPMSGVMIFGKESAGA
SNTALDNVMITDEEEIKATNPVATERFGTVAVNLQSSSTDPATGDVHVMG
ALPGMVWQDRDVYLQGPIWAKIPHTDGHFHPSPLMGGFGLKHPPPQILIK
NTPVPANPPAEFSATKFASFITQYSTGQVSVEIEWELQKENSKRWNPEVQ
YTSNYAKSANVDFTVDNNGLYTEPRPIGTRYLTRPL.

In some embodiments, the rAAV virion is an AAVrh.10 rAAV virion. The capsid many be an AAV9 capsid or functional variant thereof. In some embodiments, the AAVrh.10 capsid shares at least 98%, 99%, or 100% identity to a reference AAVrh.10 capsid, e.g.,

(SEQ ID NO: 72)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYQFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQIL
IKNTPVPADPPTTFSQAKLASFITQYSTGQVSVEIEWELQKENSKRWNPE
IQYTSNYYKSTNVDFAVNTDGTYSEPRPIGTRYLTRNL.

In some embodiments, the capsid protein is encoded by a polynucleotide supplied on a plasmid in trans to the transfer plasmid. The polynucleotide sequence of wild-type AAVrh.74 cap is as follows:

AAVrh.74 capsid coding sequence:
(SEQ ID NO: 73)
ATGGCTGCCGATGGTTATCTTCCAGATTGGCTCGAGGACAACCTCTCTGA
GGGCATTCGCGAGTGGTGGGACCTGAAACCTGGAGCCCCGAAACCCAAAG
CCAACCAGCAAAAGCAGGACAACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCC
AAGCGGGTGACAATCCGTACCTGCGGTATAATCACGCCGACGCCGAGTTT
CAGGAGCGTCTGCAAGAAGATACGTCTTTTGGGGGCAACCTCGGGCGCGC
AGTCTTCCAGGCCAAAAAGCGGGTTCTCGAACCTCTGGGCCTGGTTGAAT
CGCCGGTTAAGACGGCTCCTGGAAAGAAGAGACCGGTAGAGCCATCACCC
CAGCGCTCTCCAGACTCCTCTACGGGCATCGGCAAGAAAGGCCAGCAGCC
CGCAAAAAAGAGACTCAATTTTGGGCAGACTGGCGACTCAGAGTCAGTCC
CCGACCCTCAACCAATCGGAGAACCACCAGCAGGCCCCTCTGGTCTGGGA
TCTGGTACAATGGCTGCAGGCGGTGGCGCTCCAATGGCAGACAATAACGA
AGGCGCCGACGGAGTGGGTAGTTCCTCAGGAAATTGGCATTGCGATTCCA
CATGGCTGGGCGACAGAGTCATCACCACCAGCACCCGCACCTGGGCCCTG
CCCACCTACAACAACCACCTCTACAAGCAAATCTCCAACGGGACCTCGGG
AGGAAGCACCAACGACAACACCTACTTCGGCTACAGCACCCCCTGGGGGT
ATTTTGACTTCAACAGATTCCACTGCCACTTTTCACCACGTGACTGGCAG
CGACTCATCAACAACAACTGGGGATTCCGGCCCAAGAGGCTCAACTTCAA
GCTCTTCAACATCCAAGTCAAGGAGGTCACGCAGAATGAAGGCACCAAGA
CCATCGCCAATAACCTTACCAGCACGATTCAGGTCTTTACGGACTCGGAA
TACCAGCTCCCGTACGTGCTCGGCTCGGCGCACCAGGGCTGCCTGCCTCC
GTTCCCGGCGGACGTCTTCATGATTCCTCAGTACGGGTACCTGACTCTGA
ACAATGGCAGTCAGGCTGTGGGCCGGTCGTCCTTCTACTGCCTGGAGTAC
TTTCCTTCTCAAATGCTGAGAACGGGCAACAACTTTGAATTCAGCTACAA
CTTCGAGGACGTGCCCTTCCACAGCAGCTACGCGCACAGCCAGAGCCTGG
ACCGGCTGATGAACCCTCTCATCGACCAGTACTTGTACTACCTGTCCCGG
ACTCAAAGCACGGGCGGTACTGCAGGAACTCAGCAGTTGCTATTTTCTCA
GGCCGGGCCTAACAACATGTCGGCTCAGGCCAAGAACTGGCTACCCGGTC
CCTGCTACCGGCAGCAACGCGTCTCCACGACACTGTCGCAGAACAACAAC
AGCAACTTTGCCTGGACGGGTGCCACCAAGTATCATCTGAATGGCAGAGA
CTCTCTGGTGAATCCTGGCGTTGCCATGGCTACCCACAAGGACGACGAAG
AGCGATTTTTTCCATCCAGCGGAGTCTTAATGTTTGGGAAACAGGGAGCT
GGAAAAGACAACGTGGACTATAGCAGCGTGATGCTAACCAGCGAGGAAGA
AATAAAGACCACCAACCCAGTGGCCACAGAACAGTACGGCGTGGTGGCCG
ATAACCTGCAACAGCAAAACGCCGCTCCTATTGTAGGGGCCGTCAATAGT
CAAGGAGCCTTACCTGGCATGGTGTGGCAGAACCGGGACGTGTACCTGCA
GGGTCCCATCTGGGCCAAGATTCCTCATACGGACGGCAACTTTCATCCCT
CGCCGCTGATGGGAGGCTTTGGACTGAAGCATCCGCCTCCTCAGATCCTG
ATTAAAAACACACCTGTTCCCGCGGATCCTCCGACCACCTTCAATCAGGC
CAAGCTGGCTTCTTTCATCACGCAGTACAGTACCGGCCAGGTCAGCGTGG
AGATCGAGTGGGAGCTGCAGAAGGAGAACAGCAAACGCTGGAACCCAGAG
ATTCAGTACACTTCCAACTACTACAAATCTACAAATGTGGACTTTGCTGT
CAATACTGAGGGTACTTATTCCGAGCCTCGCCCCATTGGCACCCGTTACC
TCACCCGTAATCTGTAA.

The disclosure further provides protein sequences for AAVrh.74 VP1, VP2, and VP3, including SEQ ID NOs: 74-76, respectively, and homologs or functional variants thereof.

AAVrh.74 VP1:
(SEQ ID NO: 74)
MAADGYLPDWLEDNLSEGIREWWDLKPGAPKPKANQQKQDNGRGLVLPGY
KYLGPFNGLDKGEPVNAADAAALEHDKAYDQQLQAGDNPYLRYNHADAEF
QERLQEDTSFGGNLGRAVFQAKKRVLEPLGLVESPVKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYNFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQIL
IKNTPVPADPPTTFNQAKLASFITQYSTGQVSVEIEWELQKENSKRWNPE
IQYTSNYYKSTNVDFAVNTEGTYSEPRPIGTRYLTRNL.
AAVrh.74 VP2:
(SEQ ID NO: 75)
TAPGKKRPVEPSPQRSPDSSTGIGKKGQQPAKKRLNFGQTGDSESVPDPQ
PIGEPPAGPSGLGSGTMAAGGGAPMADNNEGADGVGSSSGNWHCDSTWLG
DRVITTSTRTWALPTYNNHLYKQISNGTSGGSTNDNTYFGYSTPWGYFDF
NRFHCHFSPRDWQRLINNNWGFRPKRLNFKLFNIQVKEVTQNEGTKTIAN
NLTSTIQVFTDSEYQLPYVLGSAHQGCLPPFPADVFMIPQYGYLTLNNGS
QAVGRSSFYCLEYFPSQMLRTGNNFEFSYNFEDVPFHSSYAHSQSLDRLM
NPLIDQYLYYLSRTQSTGGTAGTQQLLFSQAGPNNMSAQAKNWLPGPCYR
QQRVSTTLSQNNNSNFAWTGATKYHLNGRDSLVNPGVAMATHKDDEERFF
PSSGVLMFGKQGAGKDNVDYSSVMLTSEEEIKTTNPVATEQYGVVADNLQ
QQNAAPIVGAVNSQGALPGMVWQNRDVYLQGPIWAKIPHTDGNFHPSPLM
GGFGLKHPPPQILIKNTPVPADPPTTFNQAKLASFITQYSTGQVSVEIEW
ELQKENSKRWNPEIQYTSNYYKSTNVDFAVNTEGTYSEPRPIGTRYLTRN
L.
AAVrh.74 VP3:
(SEQ ID NO: 76)
MAAGGGAPMADNNEGADGVGSSSGNWHCDSTWLGDRVITTSTRTWALPTY
NNHLYKQISNGTSGGSTNDNTYFGYSTPWGYFDFNRFHCHFSPRDWQRLI
NNNWGFRPKRLNFKLFNIQVKEVTQNEGTKTIANNLTSTIQVFTDSEYQL
PYVLGSAHQGCLPPFPADVFMIPQYGYLTLNNGSQAVGRSSFYCLEYFPS
QMLRTGNNFEFSYNFEDVPFHSSYAHSQSLDRLMNPLIDQYLYYLSRTQS
TGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKD
NVDYSSVMLTSEEEIKTTNPVATEQYGVVADNLQQQNAAPIVGAVNSQGA
LPGMVWQNRDVYLQGPIWAKIPHTDGNFHPSPLMGGFGLKHPPPQILIKN
TPVPADPPTTFNQAKLASFITQYSTGQVSVEIEWELQKENSKRWNPEIQY
TSNYYKSTNVDFAVNTEGTYSEPRPIGTRYLTRNL.

In certain cases, the AAVrh.74 capsid comprises the amino acid sequence set forth in SEQ ID NO: 74. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh.74 VP1 which is set forth in SEQ ID NO: 74. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh.74 VP2 which is set forth in SEQ ID NO: 75. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh.74 VP3 which is set forth in SEQ ID NO: 76.

In some embodiments, the rAAV virion is an AAV-PHP.B rAAV virion or a neutrotrophic variant thereof, such as, without limitation, those disclosed in Int'l Pat. Pub. Nos. WO 2015/038958 A1 and WO 2017/100671 A1. For example, the AAV capsid may comprise at least 4 contiguous amino acids from the sequence TLAVPFK (SEQ ID NO: 78) or KFPVALT (SEQ ID NO: 79), e.g., inserted between a sequence encoding for amino acids 588 and 589 of AAV9.

The capsid many be an AAV-PHP.B capsid or functional variant thereof. In some embodiments, the AAV-PHP.B capsid shares at least 98%, 99%, or 100% identity to a reference AAV-PHP.B capsid, e.g.,

(SEQ ID NO: 80)
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSRT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQTLAVPFKAQAQT
GWVQNQGILPGMVWQDRDVYLQGPIWAKIPHTDGNFHPSPLMGGFGMKHP
PPQILIKNTPVPADPPTAFNKDKLNSFITQYSTGQVSVEIEWELQKENSK
RWNPEIQYTSNYYKSNNVEFAVNTEGVYSEPRPIGTRYLTRNL.

Further AAV capsids used in the rAAV virions of the disclosure include those disclosed in Pat. Pub. Nos. WO 2009/012176 A2 and WO 2015/168666 A2.

Without being bound by theory, the present inventors have determined that an AAV9 vector, AAVrh.74, or an AAVrh.10 vector will confer desirable cardiac tropism on the vector. Without being bound by theory, the present inventors have further determined that an AAV9 vector, AAVrh.74, or an AAVrh.10 vector may provide desired specificity to cardiac cells.

### Pharmaceutical Compositions and Kits

In an aspect, the disclosure provides pharmaceutical compositions comprising the rAAV virion of the disclosure and one or more pharmaceutically acceptable carriers, diluents, or excipients.

For purposes of administration, e.g., by injection, various solutions can be employed, such as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as Poloxamer 188, e.g., at 0.001% or 0.01%. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include but are not limited to sterile aqueous solutions or dispersions and sterile powders for the extemporaneous preparation of sterile injectable solutions or dispersions. In all cases the form is sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions may be prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

In another aspect, the disclosure comprises a kit comprising an rAAV virion of the disclosure and instructions for use.

### Methods of Use

In an aspect, the disclosure provides a method of increasing wildtype TNNC1 expression and/or activity in a cell, comprising contacting the cell with an rAAV of the disclosure. In another aspect, the disclosure provides a method of increasing wildtype TNNC1 expression and/or activity in a subject, comprising administering to an rAAV of the disclosure. In some embodiments, the cell and/or subject is deficient in TNNC1 messenger RNA or TNNC1 protein expression levels and/or activity and/or comprises a loss-of-function mutation in TNNC1. In certain embodiments, the cell and/or the subject has a gain of function mutation in TNNC1. In certain embodiments, the cell and/or the subject has a mutation selected from the group consisting of Y5H, A8V, L29Q, A31S, C84Y, E134D, D132N, D145E, I148V, G159D, G159R, or any combination thereof, relative to a human wildtype TNNC1 gene. The cell may be a cardiac cell, e.g. a cardiomyocyte cell.

In some embodiments, the method promotes survival of cardiac cell, e.g. a cardiomyocyte cell, in cell culture and/or in vivo. In some embodiments, the method promotes and/or restores function of the heart.

In certain embodiments, treatment with the rAAV virion results in at least two-fold, at least five-fold, at least ten-fold, or more TNNC1 protein levels detectable in cardiac fibroblasts (CFs) in the subject's heart. In certain embodiments, treatment with the rAAV virion results in at least two-fold, at least five-fold, at least ten-fold, or more TNNC1 protein levels detectable in cardiomyocytes in the subject's heart. In certain embodiments, treatment with the rAAV virion results in at least two-fold, at least five-fold, at least ten-fold, or more TNNC1 protein levels detectable in smooth muscle cells (SMCs) in the subject's heart. In certain embodiments, treatment with the rAAV virion results in at least two-fold, at least five-fold, at least ten-fold, or more TNNC1 protein levels detectable in endothelial cells (ECs) in the subject's heart. In certain embodiments, treatment with the rAAV virion results in at least two-fold, at least five-fold, at least ten-fold, or more TNNC1 protein levels detectable in the epicardium in the subject's heart. In certain embodiments, treatment with the rAAV virion results in at least two-fold, at least five-fold, at least ten-fold, or more TNNC1 protein levels detectable in the myocardium in the subject's heart. In certain embodiments, treatment with the rAAV virion results in at least two-fold, at least five-fold, at least ten-fold, or more TNNC1 protein levels detectable in the endocardium in the subject's heart

### Methods of Treatment

In another aspect, the disclosure provides a method of treating a disease or disorder in a subject in need thereof, comprising administering to the subject an effective amount of an rAAV virion of the disclosure. In some embodiments, the disease or disorder is a cardiac disease or disorder. Illustrative cardiac disorders include heart failure, TNNC1 DCM, TNNC1 HCM, arrhythmogenic right ventricular cardiomyopathy (ARVC), Brugada syndrome (BrS) and idiopathic ventricular fibrillation, left ventricular non-compaction cardiomyopathy, or restrictive cardiomyopathy, hypertrophic cardiomyopathy. In preferred embodiments, the subject suffers from or is at risk for a TNNC1-related cardiomyopathy (e.g., TNNC1 DCM or TNNC1 HCM).

The AAV-mediated delivery of TNNC1 protein to the heart may increase life span, prevent or attenuate cardiac cell degeneration, heart failure, scarring or fibrosis, reduced ejection fraction, arrythmia, exercise intolerance, angina (chest pain), dyspnea (shortness of breath), edema, left ventricular hypertrophy, left ventricular noncompaction, ventricular dilation, syncope, sudden cardiac death, exertional myalgias and cramps. The AAV-mediated delivery of TNNC1 protein to the heart may show improvement from, or prevent normal disease course detected by use of pathological electrocardiogram, echocardiogram, cardiac CT, cardiac MRI, heart biopsy, decrease in paroxysmal ventricular arrhythmias, decrease in sudden cardiac death, and/or decrease in or lack of further development of fibrosis and/or myofibrillar disarray in myocardium. The methods of the disclosure may prevent a decrease in, restore, and/or increase left ventricular ejection fraction (LVEF) and or ejection fraction, percent factional shortening, left ventricular end-systolic dimension (LVESD), and left ventricular end-diastolic dimension (LVEDD), left ventricular outflow tract velocity time integral (LVOT VTI).

In certain embodiments, the methods of disclosure result in an increase (e.g., an increase of about 5% to about 10%, about 10% to about 20%, about 20% to about 30%, about 30% to about 40%, about 40% to about 50%, about 50% to about 70%, or about 70% to about 100%) in wildtype TNNC1 protein expression in the subject. In certain embodiments, the methods of the disclosure result in an increase (e.g., an increase of about 5% to about 25%, about 25% to about 50%, about 50% to about 100%, or about 100% to about 200%) in the ratio of wildtype to mutant TNNC1 protein in the subject.

The methods disclosed herein may provide efficient biodistribution in the heart. They may result in sustained in expression in all, or a substantial fraction of, cardiac cells, e.g., cardiomyocytes. Notably, the methods disclosed herein may provide long-lasting expression of TNNC1 protein throughout the life of the subject following AAV vector administration.

Combination therapies are also contemplated by the invention. Combinations of methods of the invention with standard medical treatments (e.g., corticosteroids or topical pressure reducing medications) are specifically contemplated, as are combinations with novel therapies. In some cases, a subject may be treated with a steroid and/or combination of immune suppressing agents to prevent or to reduce an immune response to administration of a rAAV described herein.

In some embodiments, the AAV vector is administered at a dose of between about 1×10¹² and 5×10¹⁴ vector genomes (vg) of the AAV vector per kilogram (vg) of total body mass of the subject (vg/kg). In some embodiments, the AAV vector is administered at a dose of between about 1×10¹³ and 5×10¹⁴ vg/kg. In some embodiments, the AAV vector is administered at a dose of between about 5×10¹³ and 3×10¹⁴ vg/kg. In some embodiments, the AAV vector is administered at a dose of between about 5×10¹³ and 1×10¹⁴ vg/kg. In some embodiments, the AAV vector is administered at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg.

In some embodiments, the AAV vector is administered at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, or about 7×10¹⁵ vg/kg.

In some embodiments, the AAV vector is administered at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹⁵ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg.

In some embodiments, the AAV vector is administered systemically at a dose of between about 1×10¹² and 5×10¹⁴ vector genomes (vg) of the AAV vector per kilogram (vg) of total body mass of the subject (vg/kg). In some embodiments, the AAV vector is administered systemically at a dose of between about 1×10¹³ and 5×10¹⁴ vg/kg. In some embodiments, the AAV vector is administered systemically at a dose of between about 5×10¹³ and 3×10¹⁴ vg/kg. In some embodiments, the AAV vector is administered systemically at a dose of between about 5×10¹³ and 1×10¹⁴ vg/kg. In some embodiments, the AAV vector is administered systemically at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg.

In some embodiments, the AAV vector is administered systemically at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, or about 7×10¹⁵ vg/kg.

In some embodiments, the AAV vector is administered systemically at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg.

In some embodiments, the AAV vector is administered intravenously at a dose of between about 1×10¹² and 5×10¹⁴ vector genomes (vg) of the AAV vector per kilogram (vg) of total body mass of the subject (vg/kg). In some embodiments, the AAV vector is administered intravenously at a dose of between about 1×10¹³ and 5×10¹⁴ vg/kg. In some embodiments, the AAV vector is administered intravenously at a dose of between about 5×10¹³ and 3×10¹⁴ vg/kg. In some embodiments, the AAV vector is administered intravenously at a dose of between about 5×10¹³ and 1×10¹⁴ vg/kg. In some embodiments, the AAV vector is administered intravenously at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg.

In some embodiments, the AAV vector is administered intravenously at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, or about 7×10¹⁵ vg/kg.

In some embodiments, the AAV vector is administered intravenously at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg.

Evidence of functional improvement, clinical benefit or efficacy in patients may be revealed by change in New York Heart Association functional classification (NYHA Class), pathological electrocardiogram, echocardiogram, cardiac CT, cardiac MRI, heart biopsy, decrease in paroxysmal ventricular arrhythmias, decrease in sudden cardiac death, and/or decrease in or lack of further development of fibrosis in myocardium. Benefit may be observed in electrocardiographic features normally associated with dilated, hypertrophic, left ventricular non-compaction, or restrictive cardiomyopathy.

### Administration of Compositions

Administration of an effective dose of the compositions may be by routes standard in the art including, but not limited to, systemic, local, direct injection, intravenous, intracardiac administration. In some cases, administration comprises systemic, local, direct injection, intravenous, intracardiac injection. Administration may be performed by cardiac catheterization.

In some embodiments, the disclosure provides for local administration and systemic administration of an effective dose of rAAV and compositions of the invention. For example, systemic administration may be administration into the circulatory system so that the entire body is affected. Systemic administration includes parental administration through injection, infusion or implantation. Routes of administration for the compositions disclosed herein include intravenous (“IV”) administration, intraperitoneal (“IP”) administration, intramuscular (“IM”) administration, intralesional administration, or subcutaneous (“SC”) administration, or the implantation of a slow-release device, e.g., a mini-osmotic pump, a depot formulation, etc. In some embodiments, the methods of the disclosure comprise administering an AAV vector of the disclosure, or pharmaceutical composition thereof by intravenous, intramuscular, intraarterial, intrarenal, intraurethral, intracardiac, intracoronary, intramyocardial, intradermal, epidural, subcutaneous, intraperitoneal, intraventricular, ionophoretic or intracranial administration.

In particular, administration of an rAAV of the present invention may be accomplished by using any physical method that will transport the rAAV recombinant vector into the target tissue of an animal. Administration includes, but is not limited to, injection into the heart.

In some embodiments, the methods of the disclosure comprise intracardiac delivery. Infusion may be performed using specialized cannula, catheter, syringe/needle using an infusion pump. Administration may comprise delivery of an effective amount of the rAAV virion, or a pharmaceutical composition comprising the rAAV virion, to the heart. These may be achieved, e.g., via intravenous, intramuscular, intraarterial, intrarenal, intraurethral, intracardiac, intracoronary, intramyocardial, intradermal, epidural, subcutaneous, intraperitoneal, intraventricular, ionophoretic or intracranial administration. The compositions of the disclosure may further be administered intravenously.

The method of treatment disclosed herein may reduce and/or prevent one or more symptoms including but not limited to ventricular hypertrophy, syncope, chest pain, left ventricular outflow tract obstruction, left ventricular dilation, reduced ejection fraction, systolic dysfunction, NYHA Class III-IV heart failure, ventricular tachycardia, exercise intolerance, angina.

Effects of rAAV Administration

In some embodiments, administration of rAAV of the present disclosure may have beneficial effects for the subject. For example, administration of rAAV of the present disclosure may increase survivability of the subject compared to a subject that is not administered the rAAV of the present disclosure.

In some embodiments, administration of rAAV of the present disclosure increases survivability by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, or at least about 500% compared to a subject that is not administered the rAAV of the present disclosure.

In some embodiments, administration of rAAV of the present disclosure increases survivability by between 1% and 90%, between 20% and 80%, between 30% and 80%, between 40% and 80%, between 50% and 80%, between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 35%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, between 95% to 100%, between 100% to 200%, between 200% to 300%, between 300% to 400%, or between 400% to 500% compared to a subject that is not administered the rAAV of the present disclosure.

In some embodiments, administration of rAAV of the present disclosure prevents a decrease in the ejection fraction in a subject compared to a subject that is not administered the rAAV of the present disclosure. In some embodiments, administration of rAAV of the present disclosure prevents a decrease in the ejection fraction by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure.

In some embodiments, administration of rAAV of the present disclosure prevents a decrease in the ejection fraction by between 1% and 90%, between 20% and 80%, between 30% and 80%, between 40% and 80%, between 50% and 80%, between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 35%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, or between 95% to 100% compared to a subject that is not administered the rAAV of the present disclosure.

In some embodiments, administration of rAAV of the present disclosure prevents a decrease in, restore, and/or increase left ventricular ejection fraction (LVEF) in a subject compared to a subject that is not administered the rAAV of the present disclosure. In some embodiments, administration of rAAV of the present disclosure prevents a decrease in the ejection fraction by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure.

In some embodiments, administration of rAAV of the present disclosure prevents a decrease in, restore, and/or increase left ventricular ejection fraction (LVEF) by between 1% and 90%, between 20% and 80%, between 30% and 80%, between 40% and 80%, between 50% and 80%, between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 35%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, or between 95% to 100% compared to a subject that is not administered the rAAV of the present disclosure.

In some embodiments, administration of rAAV of the present disclosure prevents a decrease in, restore, and/or increase in left ventricular end-systolic dimension (LVESD) in a subject compared to a subject that is not administered the rAAV of the present disclosure. In some embodiments, administration of rAAV of the present disclosure prevents an increase in end-diastolic diameter (EDD) in a subject by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, or at least about 500% compared to a subject that is not administered the rAAV of the present disclosure.

In some embodiments, administration of rAAV of the present disclosure prevents a decrease in, restore, and/or increase in left ventricular end-systolic dimension (LVESD) in a subject by between 1% and 90%, between 20% and 80%, between 30% and 80%, between 40% and 80%, between 50% and 80%, between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 35%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, between 95% to 100%, between 100% to 200%, between 200% to 300%, between 300% to 400%, or between 400% to 500% compared to a subject that is not administered the rAAV of the present disclosure.

In some embodiments, administration of rAAV of the present disclosure prevents a decrease in, restore, and/or increase in left ventricular end-diastolic dimension (LVEDD) in a subject compared to a subject that is not administered the rAAV of the present disclosure. In some embodiments, administration of rAAV of the present disclosure prevents an increase in LVPW in a subject by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, or at least about 500% compared to a subject that is not administered the rAAV of the present disclosure.

In some embodiments, administration of rAAV of the present disclosure prevents a decrease in, restore, and/or increase in left ventricular end-diastolic dimension (LVEDD) in a subject by between 1% and 90%, between 20% and 80%, between 30% and 80%, between 40% and 80%, between 50% and 80%, between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 35%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, between 95% to 100%, between 100% to 200%, between 200% to 300%, between 300% to 400%, or between 400% to 500% compared to a subject that is not administered the rAAV of the present disclosure.

In some embodiments, administration of rAAV of the present disclosure prevents a decrease in, restore, and/or increase in left ventricular outflow tract velocity time integral (LVOT VTI) in a subject compared to a subject that is not administered the rAAV of the present disclosure. In some embodiments, administration of rAAV of the present disclosure prevents an increase in LVPW in a subject by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, or at least about 500% compared to a subject that is not administered the rAAV of the present disclosure.

In some embodiments, administration of rAAV of the present disclosure prevents a decrease in, restore, and/or increase in left ventricular outflow tract velocity time integral (LVOT VTI) in a subject by between 1% and 90%, between 20% and 80%, between 30% and 80%, between 40% and 80%, between 50% and 80%, between 1% to 2%, between 2% to 3%, between 3% to 4%, between 4% to 5%, between 5% to 6%, between 6% to 7%, between 7% to 8%, between 8% to 9%, between 9% to 10%, between 10% to 15%, between 15% to 20%, between 20% to 35%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, between 95% to 100%, between 100% to 200%, between 200% to 300%, between 300% to 400%, or between 400% to 500% compared to a subject that is not administered the rAAV of the present disclosure.

### EXAMPLES

### Example 1: Pre-Clinical Bioactivity and Efficacy

Vectors illustrated in FIGS. 1-6 are tested. AAV vectors or respective expression cassettes are tested in vitro using cultured cardiomyocytes (e.g., induced pluripotent stem cell cardiomyocytes (iPSC-CMs) from patients or primary cardiomyocytes collected from animal models) or other cells amenable to transfection or transduction with these constructs. Expression of TNNC1 is assessed by ELISA, immunofluorescence, immunohistochemistry, and Western blot. Vector DNA is detected by PCR and TNNC1 transgene mRNA is detected by qRT-PCR. Cell-based studies employing mutated cardiomyocytes reveal the benefit of overexpression of TNNC1 transgene (either following AAV vector transduction and/or transfection with vector plasmids) by normalizing Ca²⁺ binding sensitivity/dissociation kinetics and consequent normalization of contractile properties under stimulation.

Selected vectors are tested in vivo using mutant mouse models of cardiomyopathy. A D73N^{+/-} knock-in mouse model exhibits severe DCM phenotype. This mouse model exhibits one or more DCM elements of human disease. The D73N^{+/-} knock-in mouse (described in, e.g., McConnell et al. Front. Physiol. 2015; 6:242) has a mutation in the regulatory N-domain of cardiac troponin C (cTnC) which increases the rate of Ca²⁺ dissociation and reduces Ca²⁺ sensitivity. The mechanism of this phenotype is caused by the substitution of an acidic Asp (D) with neutral Asn (N) in the X position of the second Ca²⁺ binding loop. The D73N^{+/-} knock-in mouse recapitulates the DCM phenotype starting at 4 weeks with increased left ventricular (LV) size with thinner walls and fibrosis observed at 12 weeks. Further DCM phenotypes observed include left ventricular ejection fraction (LVEF) reduced to ˜28% at 4 weeks, reduced fractional shortening (FS), impaired LV systolic function, and prolonged QRS and QT intervals. The mouse model exhibits a reduction in survival by 6 weeks with a median survival of ˜12 weeks and 100% mortality by 19 weeks.

An illustrative experimental design is shown in Table 7.

TABLE 7

| ID | Group | AAV Vector | N |
|---|---|---|---|
| 1 | D73N^{+/-} | RCKT1 | 6-8 |
| 2 | D73N^{+/-} | RCKT2 | 6-8 |
| 3 | D73N^{+/-} | RCKT3 | 6-8 |
| 4 | D73N^{+/-} | RCKT4 | 6-8 |
| 5 | D73N^{+/-} | FB | 6-8 |
| | | (Negative CON) | |
| 6 | WT CON | FB | 6-8 |
| | | (Healthy CON) | |

FB = Formulation buffer;
WT = Wild Type;
CON = Control
KEY:
RCKT1 (Capsid 1, Promoter 1)
RCKT2 (Capsid 1, Promoter 1)
RCKT3 (Capsid 2, Promoter 1)
RCKT4 (Capsid 2, Promoter 2

An inducible I61Q^{+/-} knock-in mouse model exhibits moderately severe DCM phenotype. This mouse model exhibits one or more DCM elements of human disease. The I61Q^{+/-} knock-in mouse (described in, e.g., Davis et al., Cell, 2016; 165(5):1147-1159) has a doxycycline inducible cardiac single amino acid variant of cTnC. The I61Q^{+/-} knock-in mouse recapitulates the DCM phenotypes including reduced cardiac function, reduced Ca²⁺ binding and tension causing eccentric hypertrophy and LV dilation, increased diastolic LV chamber dimension, decreased septal wall thickness, increased cardiac mass, increased myocyte length-to-width ratios, and heart failure observed at ˜6 weeks. The mouse model exhibits 50% survival at ˜3 months, 25-30% survival at 4 months, and 100% mortality by 8 months.

Benefit of AAV-mediated TNNC1 expression is evidenced by increase in survival, mitigate decrease in body weight, mitigation of the normal progression of cardiomyopathy (e.g., TNNC1 DCM or TNNC1 HCM) observed on echocardiograms from left and/or right ventricle (e.g., LVESD, LVEDD), mitigation of enlarged size of right and/or left ventricle and/or mitigation of typical decrease in left ventricular ejection fraction and/or fractional shortening in the mouse models. Electrophysiological evidence of functional benefit of AAV-mediated delivery of TNNC1 protein is demonstrated by mitigation of disease-related disrupted calcium dynamics in affected cardiomyocytes, most notably on measures of L-type calcium current, sarcoplasmic reticulum calcium leak, diastolic calcium leak, as well as standard measures of calcium transients in affected (e.g., TNNC1-deficient) cardiomyocytes such as time to peak amplitude and relaxation time constants. For example, aberrant Ca²⁺ sensitivity in force generation of the myocytes may become more similar to that observed in a healthy control.

Histological analyses reveals the benefit by diminished appearance of disease-related myofiber disarray and/or fibrosis, hypertrophy, apoptotic cells, reduction in the γH2AX marker of DNA damage and reduction in disease-related change in atrial size and absolute size of heart. Additionally, benefit may also be revealed by diminished or normalized β-myosin heavy chain levels, B-type natriuretic peptide (BNP), atrial natriuretic peptide (ANP), and MYH7 levels in the myocardium relative to non AAV-TNNC1 treated, diseased controls. Benefit may also be revealed through cardiac histopathology analysis.

### Example 2: Pre-Clinical Transgene Expression

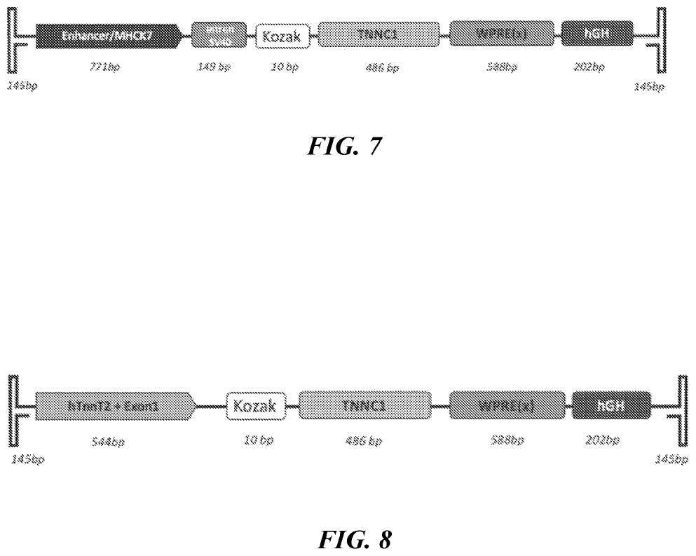

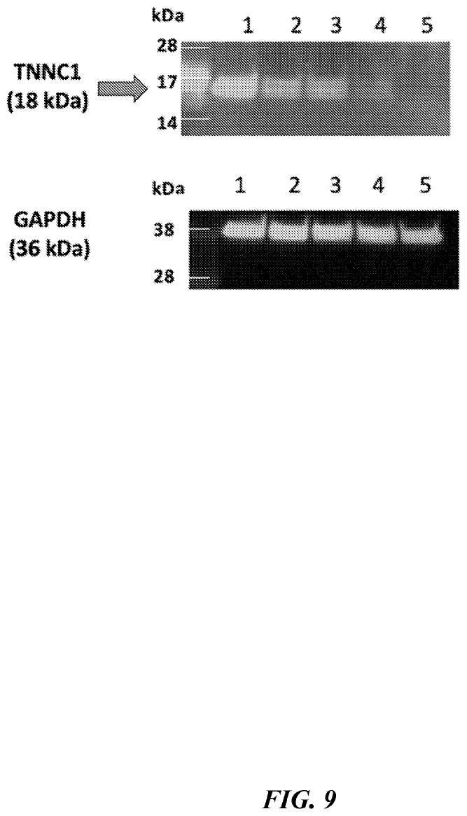

Expression cassettes illustrated in FIG. 7 and FIG. 8 were tested. AAV vectors or respective plasmid expression cassettes were tested in vitro using cultured CHO-Lec2 (mutant cells that have a 70-90% deficiency of sialic acid in their glycoproteins and gangliosides that make this cell more susceptible to AAV9 transduction). Subsequent expression of human Cardiac Troponin C transgene protein (TnC) in transduced CHO-Lec2 cells was assessed by Western blot (FIG. 9).

The vectors were also tested in vivo using a mutant mouse model of cardiomyopathy. The D73N^{+/-} knock-in mouse model exhibits a severe dilated cardiomyopathy (DCM) phenotype. This mouse model exhibits one or more DCM elements of human disease. The D73N^{+/-} knock-in mouse (described in, e.g., McConnell et al. Front. Physiol. 2015; 6:242) has a mutation in the regulatory N-domain of TNNC1 which increases the rate of Ca²⁺ dissociation and reduces Ca²⁺ sensitivity. The mechanism of this phenotype is caused by the substitution of an acidic Asp (D) with neutral Asn (N) in the X position of the second Ca²⁺ binding loop. The D73N^{+/-} knock-in mouse recapitulates the DCM phenotype starting at 4 weeks of age with increased left ventricular (LV) size, thinner ventricular walls, and subsequent fibrosis observed at 12 weeks of age. Additional markers of a DCM phenotype observed in this mouse model include reduced left ventricular ejection fraction (LVEF) by ˜28% at 4 weeks, reduced fractional shortening (FS), impaired LV systolic function, and prolonged QRS and QT intervals. This mouse model also exhibits a reduction in survival that may emerge as early as 6 weeks of life with a median survival of ˜12 weeks and 100% mortality by 21-22 weeks (a variable which may be influenced experimentally by level of stress caused by frequency of handling the animal).

The experimental design is shown in Table 8.

TABLE 8

| | | | N | |
|---|---|---|---|---|
| ID | Group | AAV Vector | Male | Female |
| 1 | D73N+/− | AAV9-MHCK7-TNNC1 | 9 | 8 |
| 2 | D73N+/− | AAV9-hTNT-TNNC1 | 11 | 3 |
| 3 | D73N+/− | AAVrh.74-MHCK7-TNNC1 | 10 | 5 |
| 4 | D73N+/− | AAVrh.74-hTNT-TNNC1 | 9 | 6 |
| 5 | D73N+/− | FB (Negative CON) | 16 | 18 |
| 6 | WT | FB (Healthy CON) | 19 | 19 |

FB = Formulation Buffer;
WT = Wild Type;
CON = Control

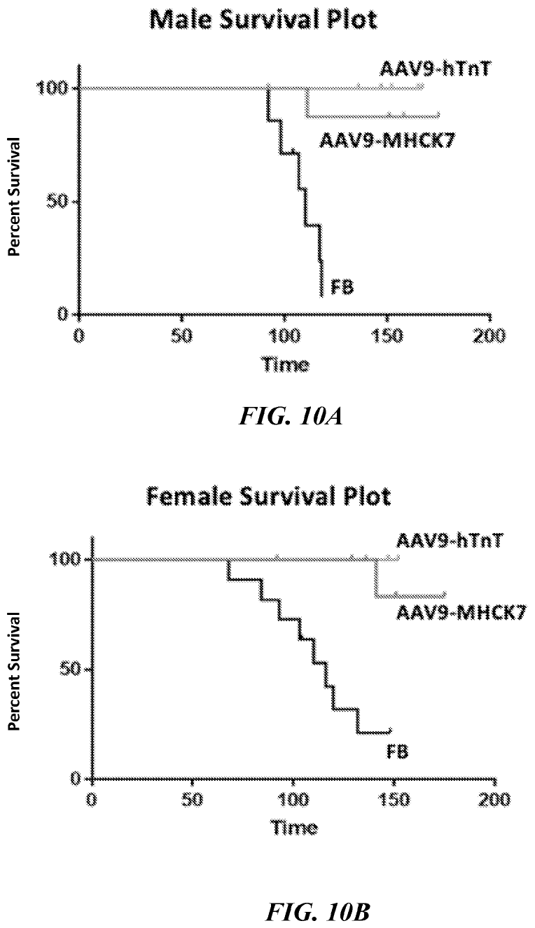

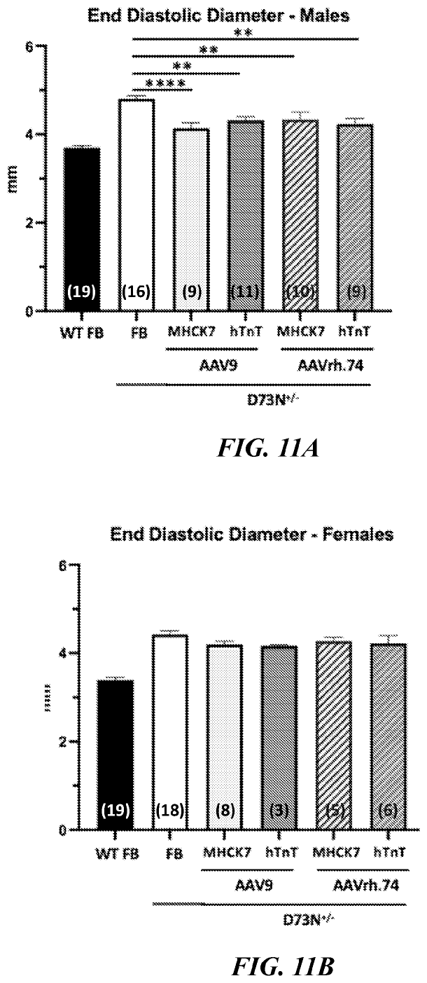

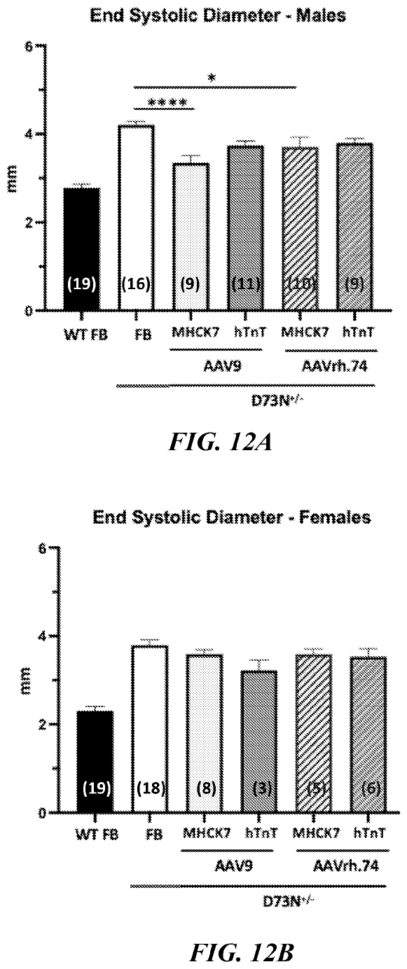

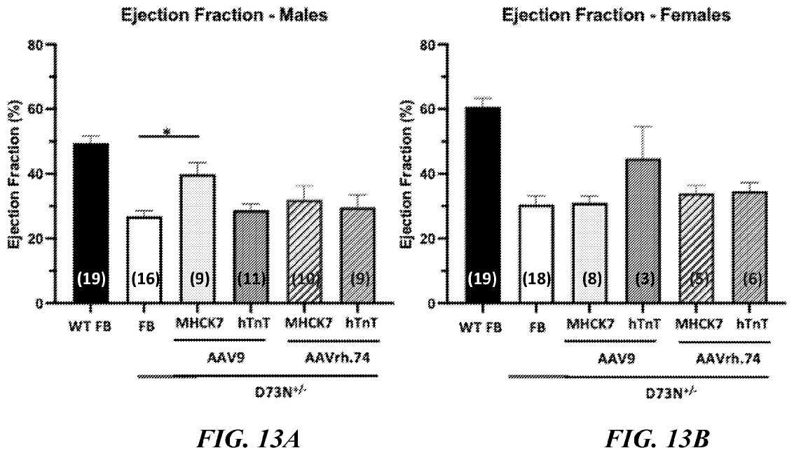

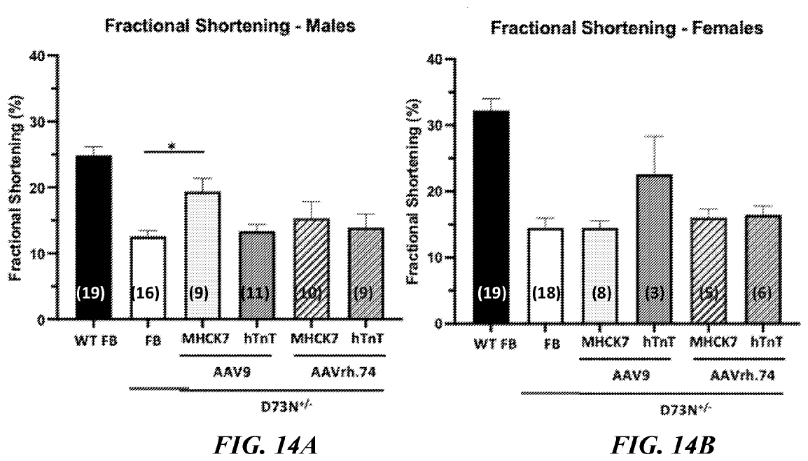

The benefit of AAV-mediated human TnC protein expression was evidenced by increase in survival. All AAV-injected animals lived considerably longer than FB injected D73N^{+/-} controls (FIG. 10A and FIG. 10B). Echocardiography revealed significant benefit of AAV-mediated overexpression of human TnC on cardiac function, evidenced by reduced end diastolic diameter (EDd) in all AAV injected male groups compared to FB injected D73N^{+/-} controls (FIG. 11A) and reduced end systolic diameter (ESd) in AAV9-MHCK7-TNNC1 and AAVrh.74-MHCK7-TNNC1 treated males compared to FB injected D73N^{+/-} controls (FIG. 12A). Mitigation of the normal progression of dilated cardiomyopathy was also revealed by significantly greater ejection fraction (FIG. 13A) and greater fractional shortening (FIG. 14A) in AAV9-MHCK7-TNNC1 injected animals relative to FB injected D73N^{+/-} controls. No gross alterations in heart weight or heart rate were observed, as expected.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A polynucleotide, comprising an expression cassette and optionally flanking adeno-associated virus (AAV) inverted terminal repeats (ITRs), wherein the polynucleotide comprises a polynucleotide sequence encoding troponin C1, cardiac type (TNNC1), or a functional variant thereof, operatively linked to a promoter.

2. The polynucleotide of claim 1, wherein the promoter is a cardiac-specific promoter.

3. The polynucleotide of claim 1 or 2, wherein the promoter is a muscle-specific promoter.

4. The polynucleotide of any one of claims 1 to 3, wherein the promoter is a cardiomyocyte-specific promoter.

5. The polynucleotide of any one of claims 1 to 4, wherein the promoter is a MHCK7 promoter.

6. The polynucleotide of claim 5, wherein the MHCK7 promoter shares at least 75%, 80%, 85%, 90%, 95%, 96%, 97%, 98%, 99% or 100% identity with SEQ ID NO: 21.

7. The polynucleotide of any one of claims 1 to 6, wherein the promoter is a cardiac troponin T (hTNNT2) promoter.

8. The polynucleotide of claim 7, wherein the hTNNT2 promoter shares at least 75%, 80%, 85%, 90%, 95%, 96%, 97%, 98%, 99% or 100% identity with SEQ ID NO: 22.

9. The polynucleotide of any one of claims 1 to 8, wherein the expression cassette comprises exon 1 of the cardiac troponin T (hTNNC1) gene, wherein optionally the hTNNT2 promoter and exon 1 together share at least 75%, 80%, 85%, 90%, 95%, 96%, 97%, 98%, 99% or 100% identity with SEQ ID NO: 23.

10. The polynucleotide of claim 1, wherein the promoter is a ubiquitous promoter, optionally a CMV promoter or a CAG promoter.

11. The polynucleotide of any one of claims 1 to 10, wherein the expression cassette comprises a polyA signal.

12. The polynucleotide of claim 11, wherein the polyA signal is a human growth hormone (hGH) polyA.

13. The polynucleotide of any one of claims 1 to 12, wherein the expression cassette comprises a Woodchuck Hepatitis Virus Posttranscriptional Regulatory Element (WPRE), optionally a WPRE(x).

14. The polynucleotide of any one of claims 1 to 13, wherein the expression cassette comprises a Kozak sequence.

15. The polynucleotide of any one of claims 1 to 14, wherein the expression cassette comprises an SV40 intron.

16. The polynucleotide of any one of claims 1 to 15, wherein the TNNC1 or functional variant thereof is TNNC1.

17. The polynucleotide of claim 16, wherein the TNNC1 is a functional TNNC1.

18. The polynucleotide of claim 16 or 17, wherein the TNNC1 is a human TNNC1.

19. The polynucleotide of any one of claims 16-18, wherein the polynucleotide comprises a TNNC1 polynucleotide sequence as set forth in SEQ ID NO: 2.

20. The polynucleotide of any one of claims 16-19, wherein the TNNC1 shares at least 75%, 80%, 85%, 90%, 95%, 96%, 97%, 98%, 99% or 100% identity with SEQ ID NO: 1.

21. The polynucleotide of any one of claims 1 to 20, wherein the polynucleotide sequence encoding TNNC1 is a human TNNC1 polynucleotide.

22. The polynucleotide of any one of claims 1 to 21, wherein the polynucleotide sequence encoding TNNC1 shares at least 75%, 80%, 85%, 90%, 95%, 96%, 97%, 98%, 99% or 100% identity with SEQ ID NO: 2.

23. The polynucleotide of any one of claims 1 to 22, wherein the polynucleotide comprises at least about 2.4 kb, at least about 2.5 kb, at least about 2.6 kb, at least about 2.7 kb, at least about 2.8 kb, at least about 3 kb, at least about 3.2 kb, at least about 3.4 kb, or at least about 3.6 kb.

24. The polynucleotide of any one of claims 1 to 23, wherein the polynucleotide comprises at most about 2.6 kb, at most about 2.7 kb, at most about 2.8 kb, at most about 3 kb, at most about 3.2 kb, at most about 3.4 kb, at most about 3.6 kb, at most about 3.8 kb, or at most about 4 kb.

25. The polynucleotide of any one of claims 1 to 24, wherein the polynucleotide comprises about 4.0 kb to 4.6 kb, about 4.0 kb to 4.5 kb, or about 4.0 kb to 4.4 kb.

26. The polynucleotide of any one of claims 1 to 25, wherein the polynucleotide comprises about 2.4 kb to 3.6 kb, about 2.5 kb to 3.5 kb, about 2.6 kb to 3.4 kb, about 2.7 kb to 3.3 kb, about 2.8 kb to 3.2 kb, or about 2.9 kb to 3.1 kb.

27. The polynucleotide of any one of claims 1 to 26, wherein the polynucleotide comprises about 2.4 kb, about 2.5 kb, about 2.8 kb, about 2.9 kb, about 3.5 kb, or about 3.6 kb.

28. The polynucleotide of any one of claim 1 to 27, wherein the polynucleotide shares at least 75%, 80%, 85%, 90%, 95%, 96%, 97%, 98%, 99% or 100% identity with any one of SEQ ID NOs: 57-62.

29. The polynucleotide of any one of claims 1 to 28, wherein the expression cassette is flanked by 5′ and 3′ inverted terminal repeats (ITRs).

30. The polynucleotide of claim 29, wherein the ITRs are AAV2 ITRs and/or the ITRs share at least 75%, 80%, 85%, 90%, 95%, 96%, 97%, 98%, 99% or 100% identity with any one of SEQ ID NO: 11-17.

31. A gene therapy vector, comprising the polynucleotide of any one of claims 1 to 30.

32. The vector of claim 31, wherein the gene therapy vector is a recombinant adeno-associated virus (rAAV) vector.

33. The vector of claim 32, wherein the rAAV vector is an AAV9 or a functional variant thereof.

34. The vector of claim 33, wherein the rAAV vector comprises a capsid protein that shares 90%, 91%, 92%, 93%, 94%, 95%, 96%, 97%, 98%, 99% or 100% identity to any one of SEQ ID NO: 70.

35. The vector of claim 34, wherein the rAAV vector is an AAVrh.74 or a functional variant thereof.

36. The vector of claim 35, wherein the rAAV vector comprises a capsid protein that shares 90%, 91%, 92%, 93%, 94%, 95%, 96%, 97%, 98%, 99% or 100% identity to any one of SEQ ID NO: 73.

37. A method of treating and/or preventing a disease or disorder in a subject in need thereof, comprising administering the vector of any one of claims 31 to 36 to the subject.

38. The method of claim 37, wherein the disease or disorder is a cardiac disorder.

39. The method of claim 37 or 38, wherein the disease or disorder is cardiomyopathy.

40. The method of claim 39, wherein the disorder is dilated cardiomyopathy.

41. The method of claim 39, wherein the disorder is left ventricular non-compaction cardiomyopathy.

42. The method of claim 39, wherein the disorder is restrictive cardiomyopathy.

43. The method of any one of claims 37 to 42, wherein the disease or disorder is heart failure.

44. The method of claim 43, wherein the disease is characterized by a low ejection fraction.

45. The method of claim 44, wherein the ejection fraction is 30% or less.

46. The method of claim 39, wherein the disorder is hypertrophic cardiomyopathy.

47. The method of claim 46, wherein the disorder is characterized by syncope, angina, and/or mild left ventricular hypertrophy.

48. The method of any one of claims 37 to 47, wherein the disease or disorder is characterized by altered calcium binding.

49. The method of any one of claims 37 to 48, wherein the disease or disorder is a cardiomyopathy associated with disfunction in TNNC1.

50. The method of any one of claims 37 to 49, wherein the disease or disorder is caused by mutation in TNNC1.

51. The method of claim 50, wherein the mutation is a gain of function mutation.

52. The method of claim 50, wherein the mutation is a loss of function mutation.

53. The method of claim 50, wherein the mutation is selected from the group consisting of Y5H, A8V, L29Q, A31S, C84Y, E134D, D132N, D145E, I148V, G159D, G159R, relative to a human TNNC1 gene.

54. The method of any one of claims 37 to 53, wherein the subject is a mammal.

55. The method of claim 54, wherein the subject is a primate.

56. The method of claim 55, wherein the subject is a human.

57. The method of any one of claim 37 to 56, wherein the vector is administered by intravenous injection, intracardiac injection, intracardiac infusion, and/or cardiac catheterization.

58. The method of any one of claims 37 to 57, wherein the administration increases wildtype TNNC1 expression by about 5% to about 10%.

59. The method of any one of claims 37 to 57, wherein the administration increases wildtype TNNC1 expression by about 10% to about 20%

60. The method of any one of claims 37 to 57, wherein the administration increases wildtype TNNC1 expression by about 20% to about 30%.

61. The method of any one of claims 37 to 57, wherein the administration increases wildtype TNNC1 expression by about 30% to about 40%.

62. The method of any one of claims 37 to 57, wherein the administration increases wildtype TNNC1 expression by about 40% to about 50%.

63. The method of any one of claims 37 to 57, wherein the administration increases wildtype TNNC1 expression by about 50% to about 70%.

64. The method of any one of claims 37 to 57, wherein the administration increases wildtype TNNC1 expression by about 70% to about 100%.

65. The method of anyone of claims 37 to 57, wherein the administration increases the ratio of wildtype to mutant TNNC1 by about 5% to about 25%.

66. The method of anyone of claims 37 to 57, wherein the administration increases the ratio of wildtype to mutant TNNC1 by about 25% to about 50%.

67. The method of anyone of claims 37 to 57, wherein the administration increases the ratio of wildtype to mutant TNNC1 by about 50% to about 100%.

68. The method of anyone of claims 37 to 57, wherein the administration increases the ratio of wildtype to mutant TNNC1 by about 100% to about 200%.

69. The method of any one of claims 37 to 68, wherein the method treats and/or prevents the disease or disorder.

70. The method of any one of claims 37 to 69, wherein the method comprises administering an effective amount of the vector.

71. The method of any one of claims 37 to 70, wherein the method comprises administering a pharmaceutical composition comprising an effective amount of the vector.

72. The method of any one of claims 37 to 71, wherein the method comprises administering between about 1×10¹¹ vector genomes and about 1×10¹⁴ vector genomes of the vector or about 1×10¹¹ vector genomes and about 1×10¹⁵ vector genomes of the vector to the subject.

73. A pharmaceutical composition comprising the vector of any one of claims 31 to 36.

74. A kit comprising the vector of any one of claims 31 to 36 or the pharmaceutical composition of claim 71 and optionally instructions for use.

75. Use of the vector of any one of claims 31 to 36 in treating a disease or disorder, optionally according to the method of any one of claims 37 to 72.

76. A vector according to any one of claims 31 to 36 for use in treating a disease or disorder, optionally according to the method of any one of claims 37 to 72.
